(19) United States
(12) Patent Application Publication
(10) Pub. No.: US 2026/0167873 A1
(43) Pub. Date: Jun. 18, 2026

(54) Catalytic Processes for Production of Olefins, Paraffins, and/or Aromatics from Mixtures of Natural Oils, and/or Fats

(71) Applicants: Gevo, Inc., Englewood, CO (US); LG Chem, Ltd., Seoul (KR)

(72) Inventors: Jonathan Smith, Highlands Ranch, CO (US); Andrew Ingram, Denver, CO (US)

(21) Appl. No.: 19/411,557

(22) Filed: Dec. 8, 2025

Related U.S. Application Data
(63) Continuation of application No. PCT/US24/41125, filed on Aug. 6, 2024.
(60) Provisional application No. 63/518,032, filed on Aug. 7, 2023.

(51) Int. Cl. C10G 11/05

(52) U.S. Cl. CPC C10G 11/05; C10G 2300/1014; C10G 2400/20; C10G 2400/22; C10G 2400/30

(57) ABSTRACT

Processes for converting one or more bio-oil(s) with or without co-feeding C₁-C₅ alcohols and/or water to one or more C₂-C₅ olefins and/or one or more aromatics are provided. In one exemplary aspect, the process can be a single stage process for the direct conversion of bio-oil(s) to olefinic (e.g., C₂-C₅) and aromatic (e.g., BTEX) mixtures carried out in a reactor using one or more catalysts that include a zeolite doped with one or more dopants and a doped or undoped alumina catalyst. Systems for carrying out these processes are also provided.

### CROSS-REFERENCE TO RELATED APPLICATION

This application claims priority to U.S. Provisional Application No. 63/518,032 filed Aug. 7, 2023, the entire contents of which are hereby expressly incorporated by reference herein.

### TECHNICAL FIELD

Systems and processes for catalytic conversion of bio-based oils, fatty acids and/or esters, and more specifically, catalytic processes resulting in the direct conversion of bio-based oils, fatty acids and/or esters to olefinic mixtures (e.g., C₂-C₅) and aromatics are provided.

### BACKGROUND

There is an increasing demand for the use of biomass for partly replacing petroleum resources for the synthesis of fuels and chemical feedstocks. The limited supply and increasing cost of crude oil and the need to reduce emission of fossil-based carbon dioxides has prompted the search for alternative processes for producing hydrocarbon products such as bio-naphtha, biodiesel, and bio-based chemical feedstocks. Made up of organic matter from living organisms, biomass is the world's leading renewable energy source. The use of bioethanol and/or fermentation-based byproducts (e.g., oils, fatty acids/esters) for the synthesis of base stocks for fuels and/or chemicals is therefore of great interest. The reaction at the root of converting ethanol to a base stock for fuels or chemicals is ethanol dehydration or dehydrogenation followed by ethylene oligomerization or aldol condensations for routes to chemicals.

Vegetable oils (i.e., bio-oils) are a common feedstock for biofuels and chemicals and may be converted into liquid fuels due to their high energy density, liquid nature and availability as a renewable raw material. Beside edible vegetable oils, which have a diversified composition in fatty acids, non-edible and used cooking oils have also received considerable attention because they do not compete with food sources.

Conversion of such bio-oils, which typically include fatty acids/esters to fuels, hydrocarbons or chemical feedstocks, often consists of a two-step process: i) hydrodeoxygenation to remove oxygen functionality followed by ii) “catalytic cracking” and/or isomerization to fuels and fuel precursors. However, using low quality and/or used cooking oil typically requires additional treatment due to high free fatty acids content and rancidity which can lead to poor quality bio-based fuels. In addition, this general approach requires two steps that result in a bio-based fuels product without the production of light olefins. Finally, the first hydrodeoxygenation step requires hydrogen adding to cost of production, followed by a second high temperature cracking step for fuel production. Accordingly, there remains a need for improved systems and methods for converting bio-oils, fatty acids/esters, or mixtures thereof to fuels, hydrocarbons, or chemical feedstock.

### SUMMARY

In certain aspects of the current subject matter, challenges associated with conversion of bio-oils can be addressed by inclusion of one or more of the features described herein or comparable/equivalent approaches as would be understood by one of ordinary skill in the art. Aspects of the current subject matter relate to processes and systems for producing one or more olefins.

Exemplary processes for converting one or more bio-oils to one or more olefins and/or aromatics are disclosed. In one exemplary aspect, the process for producing one or more olefins includes contacting an input stream with one or more catalysts in at least one reactor to form an output stream comprising the one or more olefins, the input stream comprising one or more bio-oils, and a first of the one or more catalysts comprising a first zeolite and one or first more dopants. The at least one reactor is at a temperature from about 300° C. to about 600° C., a gauge pressure from about 1 to about 10 bar, and a weight hourly space velocity (WHSV) from about 0.5 h⁻¹ to about 10 h⁻¹.

In some aspects, the at least one reactor includes two or more beds. In some aspects, the at least one reactor comprises one or more catalyst beds, wherein the process further includes impregnating at least one catalyst bed of the one or more catalysts beds with the first catalyst and the second catalyst. In some aspects, the at least one reactor can be a single bed reactor. In certain aspects, the single bed reactor can be a fixed bed reactor. In other aspects, the single bed reactor can be a fluidized bed reactor. In yet other aspects, the single bed reactor can be a moving bed reactor.

In some aspects, the one or more olefins includes C₂-C₅ olefins. In some aspects, the one or more olefins includes a predominant first olefin, wherein the predominant first olefin is propylene. In certain aspects, the one or more olefins also include ethylene. In some aspects, the one or more C₂-C₅ olefins can be present in the output stream in an amount that is from about 65 wt. % to about 85 wt. % of the total amount of unsaturated hydrocarbons present in the output stream. In certain embodiments, the one or more C₂-C₅ olefins are present in the output stream in an amount that is from about 75 wt. % to about 80 wt. % of the hydrocarbon products in the output stream.

In some aspects, the output stream also includes one or more aromatics. In certain aspects, the one or more aromatics are present in the output stream in an amount that is from about 65 wt. % to about 80 wt. % of the output stream.

In some aspects, the one or first more dopants of the first of the one or more catalysts include boron, phosphor, or a combination thereof. In certain aspects, the boron is present in the first catalyst in an amount from about 0.5 wt. % to about 3 wt. %. In one aspect, the boron is present in the first catalyst in an amount of at least about 2 wt. %. In certain aspects, the phosphor is present in the first catalyst in an amount from about 1 wt. % to about 5 wt. %. In one aspect, the phosphor is present in the first catalyst in an amount of at least about 3 wt. %.

In some aspects, the first zeolite can include a ZSM-5 zeolite.

In some aspects, the output stream can include saturates, in which a total amount of saturates can be present in the output stream does not exceed about 20 wt. % of the output stream. In certain aspects, the total amount of saturates present in the output stream can be from about 5 wt. % to 15 about wt. %. In other aspects, the total amount of saturates present in the output stream can be from about 5 wt. % to about 10 wt. %.

In some aspects, prior to contacting the input stream with the at least one catalyst, the process includes combining the input stream with water, one or more C₁-C₅ alcohols, or water and one or more C₁-C₅ alcohols. In one aspect, the one or more C₁-C₅ alcohols includes ethanol. In another aspect, the one or more C₁-C₅ alcohols includes methanol. In another aspect, the one or more C₁-C₅ alcohols includes isobutanol. In yet another aspect, the one or more C₁-C₅ alcohols includes a mixture of at least C₄ alcohols, a mixture of at least C₅ alcohols, or a mixture of at least C₄ and C₅ alcohols.

In some aspects, the one or more bio-oils are produced by fermentative processes.

In some aspects, the process can include removing at least a portion of C₂ olefins from the output stream.

In some aspects, the process can include removing at least a portion of C₄ olefins from the output stream.

In some aspects, the process can include removing at least a portion of C₅ olefins from the output stream.

In some aspects, the process can include removing at least a portion of aromatics from the output stream.

In some aspects, the temperature can be from about 350° C. to about 500° C. In other aspects, the temperature can be from about 450° C. to about 500° C.

In some aspects, the WHSV can be from about 2.0 h⁻¹ to about 5.0^{h-1}. In other aspects, the WHSV can be from about 3.0 h⁻¹ to about 5.0 h⁻¹.

In some aspects, a second of the one or more catalysts can be a second zeolite and one or more second dopants. In certain aspects, the first zeolite and the second zeolite are the same or different, and the one or more first dopants and the one or more second dopants are the same or different.

In some aspects, a second of the one or more catalysts can be a doped or undoped alumina catalyst. In certain aspects, the doped alumina catalyst includes, in neutral or ionic form, zirconium, titanium, tungsten, silicon, fluorine, or any combination thereof. In certain aspects, the second of the one or more catalysts can be a silicated [symbol] -alumina, zirconated [symbol] -alumina, titanated [symbol] -alumina, niobium [symbol] -alumina, or fluorinated [symbol] -alumina, an undoped [symbol] -alumina, an undoped zeolite, a silica alumina catalyst, or any combination thereof.

In another exemplary process for producing olefins, the process includes contacting an input stream in a single reactor with at least a first catalyst and a second catalyst to form an output stream comprising the one or more olefins, the input stream including one or more bio-oils, and the single reactor being at a temperature about 300° C. to about 600° C., a gauge pressure from 1 to about 10 bar, and a weight hourly space velocity (WHSV) from about 0.5 h⁻¹ to about 10 h⁻¹. The first catalyst can be a first zeolite and one or more first dopants.

In some aspects, the second catalyst can be a second zeolite and one or more second dopants. In certain aspects, the first zeolite and the second zeolite are the same or different, and the one or more first dopants and the one or more second dopants are the same or different.

In some aspects, the second catalyst can be a doped or undoped alumina catalyst. In certain aspects, the doped alumina catalyst can be, in neutral or ionic form, zirconium, titanium, tungsten, silicon, fluorine, or any combination thereof. In certain aspects, the second catalyst can be a silicated [symbol] -alumina, zirconated [symbol] -alumina, titanated [symbol] -alumina, niobium [symbol] -alumina, or fluorinated [symbol] -alumina, an undoped [symbol] -alumina, an undoped zeolite, a silica alumina catalyst, or any combination thereof.

In some aspects, the single reactor includes one or more catalyst beds, and the process further includes impregnating at least one catalyst bed of the one or more catalysts beds with the first catalyst and the second catalyst. In certain aspects, the one or more catalyst beds are stacked relative to each other within the single reactor.

In some aspects, prior to contacting the input stream, the process includes adding the one or more first dopants to the first catalyst. In some aspects, prior to contacting the input stream, the process includes adding the one or more second dopants to the second catalyst.

In some aspects, the one or more olefins includes C₂-C₅ olefins. In some aspects, the one or more olefins includes a predominant first olefin, wherein the predominant first olefin is propylene. In certain aspects, the one or more olefins also include ethylene. In some aspects, the one or more C₂-C₅ olefins can be present in the output stream in an amount that is from about 65 wt. % to about 85 wt. % of the total amount of unsaturated hydrocarbons present in the output stream. In certain embodiments, the one or more C₂-C₅ olefins are present in the output stream in an amount that is from about 75 wt. % to about 80 wt. % of the hydrocarbon products in the output stream.

In some aspects, the output stream also includes one or more aromatics. In certain aspects, the one or more aromatics are present in the output stream in an amount that is from about 65 wt. % to about 80 wt. % of the output stream.

In some aspects, the one or first more dopants of the first catalyst include boron, phosphor, or a combination thereof. In certain aspects, the boron is present in the first catalyst in an amount from about 0.5 wt. % to about 3 wt. %. In one aspect, the boron is present in the first catalyst in an amount of at least about 2 wt. %. In certain aspects, the phosphor is present in the first catalyst in an amount from about 1 wt. % to about 5 wt. %. In one aspect, the phosphor is present in the first catalyst in an amount of at least about 3 wt. %.

In some aspects, the first zeolite can include a ZSM-5 zeolite.

In some aspects, the output stream can include saturates, in which a total amount of saturates can be present in the output stream does not exceed about 20 wt. % of the output stream. In certain aspects, the total amount of saturates present in the output stream can be from about 5 wt. % to 15 about wt. %. In other aspects, the total amount of saturates present in the output stream can be from about 5 wt. % to about 10 wt. %

In some aspects, prior to contacting the input stream with the first catalyst, the process includes combining the input stream with water, one or more C₁-C₅ alcohols, or water and one or more C₁-C₅ alcohols. In one aspect, the one or more C₁-C₅ alcohols includes ethanol. In another aspect, the one or more C₁-C₅ alcohols includes methanol. In another aspect, the one or more C₁-C₅ alcohols includes isobutanol. In yet another aspect, the one or more C₁-C₅ alcohols includes a mixture of at least C₄ alcohols, a mixture of at least C₅ alcohols, or a mixture of at least C₄ and C₅ alcohols.

In some aspects, the one or more bio-oils are produced by fermentative processes.

In some aspects, the process can include removing at least a portion of C₂ olefins from the output stream.

In some aspects, the process can include removing at least a portion of C₄ olefins from the output stream.

In some aspects, the process can include removing at least a portion of C₅ olefins from the output stream.

In some aspects, the process can include removing at least a portion of aromatics from the output stream.

In some aspects, the temperature can be from about 350° C. to about 500° C. In other aspects, the temperature can be from about 450° C. to about 500° C.

In some aspects, the WHSV can be from about 2.0 h⁻¹ to about 5.0^{h-1}. In other aspects, the WHSV can be from about 3.0 h⁻¹ to about 5.0 h⁻¹.

In yet another exemplary process for producing olefins, the process includes contacting an input stream in at least one reactor with at least a first catalyst and a second catalyst to form an output stream comprising the one or more olefins, the input stream including one or more bio-oils, the first catalyst comprises a zeolite and two dopants, and the second catalyst includes a doped or undoped alumina catalyst. The at least one reactor is at a temperature from about 450° C. to about 500° C., a gauge pressure from about 1 to about 2 bar, and a weight hourly space velocity (WHSV) from about 2.0 h⁻¹ to about 5.0 h⁻¹.

In some aspects, the first of the two dopants can be boron and the second dopant can be phosphor. In certain aspects, the boron is present in the first catalyst in an amount of at least about 2 wt. % and the phosphor is present in the catalyst in an amount of at least about 3 wt. %.

In some aspects, the zeolite can include a ZSM-5 zeolite.

In some aspects, the doped alumina catalyst can be a silicated [symbol] -alumina, zirconated [symbol] -alumina, titanated [symbol] -alumina, niobium [symbol] -alumina, or fluorinated [symbol] -alumina, an undoped [symbol] -alumina, an undoped zeolite, a silica alumina catalyst, or any combination thereof.

In some aspects, prior to contacting the input stream with the first catalyst and second catalyst, the process includes combining the input stream with water, one or more C₁-C₅ alcohols, or water and one or more C₁-C₅ alcohols.

In one aspect, the one or more C₁-C₅ alcohols includes ethanol. In another aspect, the one or more C₁-C₅ alcohols includes methanol. In another aspect, the one or more C₁-C₅ alcohols includes isobutanol. In yet another aspect, the one or more C₁-C₅ alcohols includes a mixture of at least C₄ alcohols, a mixture of at least C₅ alcohols, or a mixture of at least C₄ and C₅ alcohols.

In some aspects, the one or more bio-oils are produced by fermentative processes.

In some aspects, the process can include removing at least a portion of C₂ olefins from the output stream.

In some aspects, the process can include removing at least a portion of C₄ olefins from the output stream.

In some aspects, the process can include removing at least a portion of C₅ olefins from the output stream.

In some aspects, the process can include removing at least a portion of aromatics from the output stream.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter of this disclosure are contemplated as being part of the inventive subject matter disclosed herein. It should also be appreciated that terminology explicitly employed herein that also can appear in any disclosure incorporated by reference should be accorded a meaning most consistent with the particular concepts disclosed herein.

### BRIEF DESCRIPTION OF THE DRAWINGS

The accompanying drawings, which are incorporated into and constitute a part of this specification, show certain aspects of the subject matter disclosed herein and, together with the description, help explain some of the principles associated with the disclosed aspects. In the drawings:

FIG. 1 is a graphical diagram showing a typical composition of gaseous products associated with co-cracking of bio-oils with ethanol using previous processes.

FIG. 2 is a schematic illustration of an exemplary system for conversion of bio-oil(s) to olefins and aromatics, where the first input feed includes one or more bio-oil(s) with an optional co-feed of water and/or one or more C₁-C₅ alcohols.

### DETAILED DESCRIPTION

In the following description, certain specific details are set forth in order to provide a thorough understanding of various aspects. However, one skilled in the art will understand that the disclosure can be practiced without these details. In other instances, well-known structures have not been shown or described in detail to avoid unnecessarily obscuring descriptions of the aspects. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed disclosure.

Reference throughout this specification to “one aspect” or “an aspect” means a particular feature, structure or characteristic described in connection with the aspect is included in at least one aspect. Thus, the appearances of the phrases “in one aspect” or “in an aspect” in various places throughout this specification are not necessarily all referring to the same aspect. Furthermore, the particular features, structures, or characteristics can be combined in any suitable manner in one or more aspects. Also, as used in this specification and the appended claims, the singular forms “a,” “an,” and “the” include plural referents unless the context clearly dictates otherwise. It should also be noted that the term “or” is generally employed in its sense including “and/or” unless the context clearly dictates otherwise.

The word “about” when immediately preceding a numerical value means a range of plus or minus 10% of that value, e.g., “about 50” means 45 to 55, “about 25,000” means 22,500 to 27,500, etc. Furthermore, the phrases “less than about” a value or “greater than about” a value should be understood in view of the definition of the term “about” provided herein.

“WHSV” refers to weight hourly space velocity and is defined as the weight of the feed flowing per unit weight of the catalyst per hour.

As used herein, “unsaturated hydrocarbons” are organic compounds that are entirely made up of carbon and hydrogen atoms and consist of a double or a triple bond between two adjacent carbon atoms. For example, unsaturated hydrocarbons include olefins, diolefins, and alkynes.

“Aromatics” or “aromatic compounds” as used herein refer to any of a large class of unsaturated organic chemical compounds characterized by containing one or more planar rings of carbon atoms joined by covalent bonds of two different kinds (e.g., benzene, naphthalene, etc.).

“Trace amounts” or “trace levels” as used herein refer to levels less than 2%. In some aspects, trace amounts or trace levels can refer to levels less than about 1.5%, less than about 1%, less than about 0.5%, less than about 0.1%, from about 0.1% to about 1.8%, or from about 1% to about 1.5%.

“Single stage transformation” refers to processes which occur within a single reactor system.

“Saturates” as used herein refer to one or more C₂-C₅ paraffins. In some aspects, saturates can include ethane, propane, butanes, pentanes, or any combination thereof.

All yields and conversions described herein are on a weight basis unless specified otherwise.

Using biomass pyrolysis oil (e.g., bio-oil) to partially substitute fossil fuels is essential to relieve the shortage of traditional transport fuels. Crude bio-oil has a high oxygen content and high water content, which leads to a variety of inferior properties such as a low heating value, strong corrosiveness and a high viscosity. Therefore, upgrading the crude bio-oil is a required step for its high-grade utilization. Through catalytic cracking over acidic zeolites, the oxygen in bio-oil can be removed in the forms of CO, CO₂ and H₂O. Therefore, the oxygenated bio-oil can be transformed into liquid fuels rich in aromatic hydrocarbons. Aromatic hydrocarbons can be used to produce commercial gasoline as well as important chemicals such as benzene, toluene and xylenes (BTX). Different zeolites have been tested in the study of bio-oil cracking and HZSM-5 was found to have the best performance, corresponding to the highest hydrocarbon yield, followed by HY and silica-alumina, while only a few hydrocarbons were produced over silicalite and H-mordenite. It has been observed that HZSM-5 was superior for aromatic hydrocarbon production than HY during bio-oil cracking. Additionally, a Nickel-modified HZSM-5 catalyst was developed and used for bio-oil cracking. However, it was observed that the catalysts used in these bio-oil cracking processes were quickly and severely deactivated resulting in low yields of the liquid hydrocarbons. Without being bound by theories, two reasons may be responsible for this observation: (1) There are some large molecular sugar and phenol oligomers found in bio-oil, which are non-volatile and easily coked, indicating a requirement of separation pretreatment; and (2) Due to the high oxygen content and unsaturation of the components of bio-oil, the corresponding effective hydrogen to carbon ratios are low. Consequently, products with low hydrogen-to-carbon ratios like coke are favored in the cracking process. As used herein, “coke” refers to high molecular weight and high boiling carbon deposits formed during processes and that may be present on the catalyst. Such coke species constitute a loss in yield and may deactivate the catalyst in the process.

Therefore, to suppress the formation of coke during bio-oil cracking, it is necessary to increase the integral hydrogen-to-carbon ratios of the feedstock, for example, by introducing co-cracking reactants with high hydrogen-to-carbon ratios into the reaction. Two kinds of compounds are suitable for co-cracking: (1) Fluid catalytic cracking (FCC) feedstock gas-oil, and (2) aliphatic alcohols. Previous studies on co-cracking of a bio-oil model compound mixture and gas-oil showed that the introduction of gas-oil could benefit the conversion of the model compounds into liquid and gaseous hydrocarbons. Other studies reported a co-cracking process using hydrodeoxygenated bio-oil with gas-oil, and achieved a high gasoline yield of above 40 wt %. Aliphatic alcohols, for example methanol and ethanol, show superior reactivity in processes similar to catalytic cracking and therefore have potential to be used in the co-cracking process. In further studies, the introduction of methanol was found to prolong the lifetime of the catalyst in the cracking of bio-oil model compounds.

In other studies, the co-cracking of a distilled fraction from bio-oil molecular distillation and ethanol was found to have enriched the ketones and acids in bio-oil with no large molecular sugar and phenol oligomers, which resulted in its higher reactivity when compared to crude bio-oil. It was found that the oil phase yield was as high as 25.9 wt % and the hydrocarbon (mainly aromatics) content in this oil phase reached 98.3%. However, the selectivity for C₃-C₄ hydrocarbons was also high, which shows great potential for an increase in the yield of aromatic hydrocarbons by promoting aromatization during the cracking process.

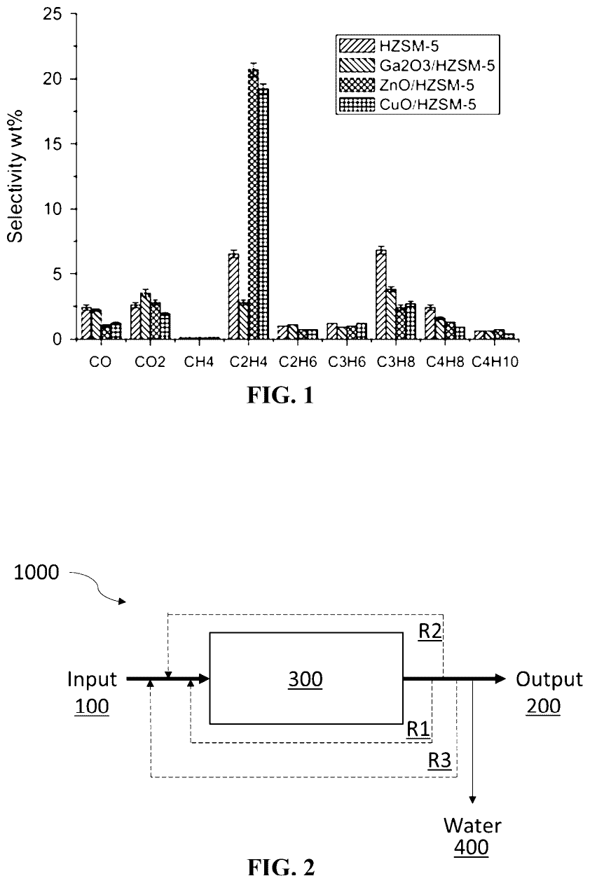

The ability to crack bio-oils as a single feedstock, or via co-cracking a bio-oil feed with bio-based aliphatic alcohols that results in a significant fraction of i) C₂-C₄ olefins and ii) aromatics without ex-situ hydrogen in a single step provides a unique economic advantage as compared to the classical two-step process (i.e., hydrodeoxygenation and cracking) for upgrading bio-oils to primarily aromatics. FIG. 1 represents a typical composition of gaseous products associated with co-cracking of bio-oils with ethanol using previous processes. Paraffin content of the C₂-C₄ hydrocarbon fraction is extremely high as compared to the C₂-C₄ olefin content.

Aspects of the subject matter disclosed herein provide processes in which a catalyst system is used to convert bio-oils, fatty acids/esters, or mixtures thereof to olefinic mixtures, including a predominant first olefin, in high yield with low levels of saturates at competitive costs. Consistent with the current disclosure, processes for the single step conversion of one or more bio-oils, fatty acids/esters, or mixtures thereof to olefinic mixtures (e.g., C₂-C₅) with low levels of saturates can be carried out in a single reactor (e.g., using a single catalyst bed or stacked catalyst beds, impregnated with at least one catalyst). The C₂-C₅ olefins can be easily oligomerized to base stocks used in the production of fuels in high yields.

As used herein, “bio-oils” includes triglycerides, lipids/fats or derivatives thereof. In some aspects, the one or more of bio-oils or mixtures thereof include edible and non-edible (e.g., used) vegetable oils, such as corn oil, soybean oil, olive oil, peanut oil, rapeseed oil, including canola, coconut oil, palm oil, or animal based products such as tallow or lard, among others. In some aspects, the bio-oils, fatty acids/esters, or mixtures thereof may be subjected to partial or complete hydrogenation, and/or the olefin isomerized prior to use as feed. In some aspects, the bio-oils include fatty acids/esters derived from triglycerides, for example, by hydrolysis of triglycerides to fatty acids or transesterification with alcohols to fatty acid esters. In some aspects, the bio-oils or mixtures thereof can be produced by fermentative processes. Table 1 provides compositional variations for common triglyceride bio-oils.

TABLE 1

| | Symbol | Cotton- | Coconut | Corn | Palm | Peanut | Palm | Linseed | Rice | Rape- | Olive |
|---|---|---|---|---|---|---|---|---|---|---|---|
| Saturated | | | | | | | | | | | |
| Caproic | 6:0 | | 0.4 | | 0.2 | | | | | | |
| Caprylic | 8:0 | | 7.3 | | 3.3 | | | | | | |
| Capric | 10:0 | | 6.6 | | 3.5 | | | | | | |
| Lauric | 12:0 | | 47.8 | | 47.8 | | 0.2 | | | | |
| Myristic | 14:0 | 0.9 | 18.1 | | 16.3 | 0.3 | 1.1 | | 0.4 | | 0.02 |
| Palmitic | 16:0 | 24.7 | 8.9 | 10.9 | 8.5 | 11.6 | 44.1 | 6.0 | 19.8 | 3.9 | 10.5 |
| Margaric | 17:0 | | | | | | | | | | 0.05 |
| Stearic | 18:0 | 2.3 | 2.7 | 1.8 | 2.4 | 3.1 | 4.4 | 2.5 | 1.9 | 1.9 | 2.6 |
| Arachidic | 20:0 | 0.1 | 0.1 | | | 1.5 | 0.2 | 0.5 | 0.9 | 0.6 | 0.4 |
| Behenic | 22:0 | | | | | 3.0 | | | 0.3 | 0.2 | 0.2 |
| Lignoceric | 24:0 | | | | | 1.0 | | | | 0.2 | 0.1 |
| TOTAL | | 28.0 | 91.9 | 22.7 | 82.0 | 20.3 | 50 | 9.0 | 23.3 | 6.8 | 13.87 |
| Unsaturated | | | | | | | | | | | |
| Myristoleic | 14:3 w-5 | | | | | | | | | | |
| Palmitoleic | 16:1 w-7 | 0.7 | | | | 0.5 | | 0.1 | 0.2 | | 0.6 |
| Heptadecenoic | 17:1 w-15 | | | | | | | | | | 0.09 |
| Oleic | 18:1 w-9 | 17.6 | 6.4 | 24.2 | 15.4 | 38.0 | 37.5 | 19.0 | 42.3 | 64.1 | 76.9 |
| Linoleic | 18:2 w-6 | 53.3 | 1.6 | 58.0 | 2.4 | 41.0 | 10 | 24.1 | 31.9 | 18.7 | 7.5 |
| Linolenic | 18:3 w-3 | 0.3 | | 0.7 | | | | 47.4 | 1.2 | 9.2 | 0.6 |
| Gadolenic | 20:1 w-9 | | | | | 1.0 | 0.5 | | 0.5 | 1.0 | 0.3 |
| TOTAL | | 72.0 | 18.1 | 77.3 | 18.0 | 79.7 | 50 | 91 | 76.7 | 93.2 | 86.13 |
| Polyunsaturated | | | | | | | | | | | |
| Ricinoleic | 18 | | | | | | | | | | |
| Rosin acids | — | | | | | | | | | | |
| % FFA | | 0.5- | 1.0- | 1.7 | 0.1 | 0.8 | 2- | 2 | 5- | 0.5- | 0.5- |
| | | 0.6 | 3.5 | | | | 14 | | 15 | 3.8 | 3.3 |

| | Soy- | Sun- | Linola | Lard | Butterfat | Tallow | Tall | Castor | Jatropha |
|---|---|---|---|---|---|---|---|---|---|
| Saturated | | | | | | | | | |
| Caproic | | | | | 2 | | | | |
| Caprylic | | | | | 2 | | | | |
| Capric | | | | | 3 | | | | |
| Lauric | | 0.5 | | 0.5 | 3.5 | | | | |
| Myristic | 0.1 | 0.2 | | 1.5 | 1.1 | 3 | | | |
| Palmitic | 11.0 | 6.8 | 5.6 | 26 | 26 | 26 | 2 | 1.0 | 14.6 |
| Margaric | | | | 0.5 | | 0.5 | | | |
| Stearic | 4.0 | 4.7 | 4.0 | 13.5 | 11 | 22.5 | 1 | 1.0 | 7.4 |
| Arachidic | 0.3 | 0.4 | | | 2 | | 0.5 | | |
| Behenic | 0.1 | | | | | | | | |
| Lignoceric | | | | | | | | | |
| TOTAL | 5.5 | 12.6 | 9.6 | 42.0 | 60.5 | 52.0 | 3.5 | 2.0 | 22.0 |
| Unsaturated | | | | | | | | | |
| Myristoleic | | | | | | 0.5 | | | |
| Palmitoleic | 0.1 | 0.1 | | 4 | 2 | 2.5 | | | 0.8 |
| Heptadecenoic | | | | 0.5 | 3 | 0.5 | | | |
| Oleic | 23.4 | 18.6 | 15.9 | 43 | 26 | 43 | 16 | 3.0 | 47.5 |
| Linoleic | 53.2 | 68.2 | 71.8 | 9 | 2.5 | 1.5 | 20 | 4.2 | 28.7 |
| Linolenic | 7.8 | 0.5 | 2.0 | 0.5 | 4 | | | 0.3 | 1.0 |
| Gadolenic | | | | 1 | | | 0.5 | | |
| TOTAL | 84.5 | 87.4 | 90.4 | 58.0 | 37.5 | 48.0 | 54.5 | 7.5 | 78.0 |
| Polyunsaturated | | | | | 2 | | 4 | | |
| Ricinoleic | | | | | | | | 89.5 | |
| Rosin acids | | | | | | | 40 | | |
| % FFA | 0.3- | 0.1- | 0.3 | 0.5 | | 5- | | | |
| | 1.6 | 1.5 | | | | 20 | | | |

In some aspects, the processes described herein can be carried out in a single bed reactor (e.g., fixed bed reactor, fluidized bed, or moving bed). In other aspects, the processes described herein can be carried out in a single stacked bed reactor. For example, in certain aspects, bio-oils, fatty acids/esters, or mixtures thereof (e.g., corn oil), can be converted to olefinic mixtures (e.g., C₂-C₅) in a single reactor having a first catalyst in the top section of the reactor with a second catalyst being located in a section of the reactor below the first catalyst. In either a single-stage process (e.g., using a single bed reactor, e.g., with a mixed catalyst bed) or in a two-stage process (e.g., using a stacked bed reactor where each catalyst bed is impregnated with at least one catalyst), the resulting C₂-C₅ olefinic mixture is suitable for oligomerization into either gasoline, jet, or diesel fuel cuts at relatively low temperatures and pressures depending upon the oligomerization catalyst selected. Further, in some aspects, the single bed reactor or stacked bed reactor can be defined as a fixed bed reactor, whereas in other aspects, a fluidized bed reactor can be used.

Systems and processes for catalytic conversion of bio-oils, fatty acids/esters, or mixtures thereof (e.g., corn oil with aliphatic alcohols) are provided. In general, a catalytic process consistent with the present disclosure includes dehydration, dehydrogenation, skeletal carbon build-up, “cracking,” and aromatization resulting in high carbon yields to low molecular weight olefins (e.g., C₂-C₅) and aromatics (e.g., Benzene, Toluene, Ethylbenzene and Xylenes, commonly referred to as “BTEX”). Further, such catalytic processes can result in low levels of saturates (e.g., no greater than about 20 wt % of the output stream for a given conversion of bio-oil(s) to their corresponding olefins and such corresponding olefins to other hydrocarbons). In this single stage process, the catalyst mixture can result in a C₂-C₅ olefinic mixture providing access to low molecular weight olefins in yields with good carbon accountability as defined by moles of carbon fed into the system as bio-oil(s) versus moles of carbon out of the system incorporated in the C₂-C₅ olefinic and aromatic mixture. Furthermore, use of recycle streams of specific olefins (e.g., C₂-C₅) advantageously results in the ability to maximize the formation of desirable olefins such as propylene, butenes, or mixtures thereof, as well as BTEX. In some aspects, the mixture of olefins are suitable for oligomerization to either gasoline, jet, or diesel fuel cuts at relatively low temperatures and pressures depending upon the oligomerization catalyst selected.

To address the challenges described above, and to define an approach to convert bio-oil(s) into a viable feedstock resulting in high yields to fuels, a process has been developed capable of converting bio-oil(s), via a single unit operation (e.g., in a single reactor or a single reactor section without intermediate separation), to a mixture of C₂-C₅ olefins including a predominant olefin, and optionally an aromatic fraction consisting mostly of biobased BTEX components, in high yield, which is readily separable for use as chemical feedstocks or easily oligomerized to base stocks for fuels in high yield. The ability to accomplish numerous unit operations and chemical transformations in a single reaction process, as presented herein, provides the practitioner with favorable economics due to reduced fixed and variable costs, lower capital investment, less energy, increased productivity, and maximized site profitability. Thus, unlike current systems and methods, the present process described herein is one-step, produces both precursors to fuels and value-added chemical building blocks, and eliminates the need for hydrogen.

To this end, consistent with the current disclosure the conversion of bio-oil(s) and, optionally, a co-feed of water and/or one or more C₁-C₅ alcohols (e.g., methanol, ethanol, isobutanol, a mixture of at least C₄ and/or C₅ alcohols, or any combination thereof), proceeds similarly to a C₂-C₅ olefin mixture in high yield and carbon accountability. Without being bound to theory, an exemplary single reaction step encompasses i) dehydration of alcohols to ethers and/or olefins, ii) oligomerization of light olefins, especially ethylene, to C₄₊ olefins, iii) skeletal rearrangement, iv) cracking of larger olefins and alkylated aromatic species to light olefins (e.g., ethylene, propylene, butenes) along with C₅₊ olefins, v) hydrogen transfer leading to minor amounts of aromatics and saturates, vi) alkylation of said aromatic species by olefins, alcohols, and/or ethers, and vii) decarboxylation and/or decarbonylation of ester and acid functionalities. Thus, passing a vaporized stream of one or more bio-oil(s) and, optionally, a co-feed of water and/or C₁-C₅ alcohols (e.g., methanol, ethanol, isobutanol, a mixture of at least C₄ and/or C₅ alcohols, or any combination thereof), over a single fixed catalyst bed containing a physical mixture of containing, optionally, a first part of a doped zeolite (e.g., zeolite doped with boron, phosphor, and optionally, one or more additional dopants) combined with a second catalyst at between about 300° C. to about 600° C. results in a C₂-C₅ olefin mixture, and optionally aromatics, which can be separated for sale, or after removal of condensed water, oligomerized “as-is” to primarily jet and/or diesel fuel. In some aspects, the second catalyst may be the same or a different doped zeolite. In some aspects, the second catalyst may be a silicated, zirconated, titanated, niobium, or fluorinated [symbol] -alumina.

Furthermore, the present systems and processes can optionally include the recycle of one or more specific olefin fractions (e.g., C₂+C₄+C₅ or C₂+C₅, etc.) and/or water in a closed-loop process configuration, while co-feeding the bio-oil(s).

This can result in the maximization of yields to selected olefins and/or aromatics. For example, the recycle of the C₂+C₄+C₅ olefin fraction in combination with co-feeding bio-oil(s) using the present system and processes provided results in improved propylene carbon yield. Selective recycle of the C₂+C₅ olefin fraction can result in improved propylene and butenes combined yield. Additionally, recycle of the C₄+C₅ olefin fraction can result in an improved ethylene and propylene combined yield. Recycling the olefin fraction of choice enables olefin production for chemicals and/or fuels production. An exemplary single-step reaction can encompass i) in-situ dehydration of alcohols to ethers and or olefins, ii) oligomerization of light olefins, especially ethylene, to C₄₊ olefins, iii) skeletal rearrangement, iv) cracking of larger olefins and alkylated aromatic species to light olefins (e.g., ethylene, propylene, butenes) along with C₅₊ olefins, v) hydrogen transfer leading to minor amounts of aromatics and saturates, vi) alkylation of said aromatic species by olefins, alcohols, and/or ethers, and vii) decarboxylation and/or decarbonylation of ester and acid functionalities. Recycling the olefin fraction of choice and/or any water produced or optionally co-fed can therefore enable targeted olefinic and/or aromatic production for chemicals and/or fuels production.

Unlike conversion of ethylene, propylene and other olefins of higher molecular weight (C₄₊) can easily be oligomerized over a wide range of catalysts of both zeolitic and non-zeolitic type. The present disclosure, enabling the ability to convert bio-oil(s) in a single stage, or two-stage reactor configuration in series, to an olefin mixture which includes of primarily C₂-C₅ olefins with low levels of saturates, presents a path towards an economical process to convert bio-oil(s) to base stocks for chemicals and/or fuels. The process according to the invention implements a scheme that includes a “single” stage transformation of an aqueous mixture of one or more bio-oil(s) into primarily an olefinic mixture, including a predominant first olefin, which can be separated to isolate key low molecular weight olefins used throughout the industry as chemical building blocks, or can be easily oligomerized in high yield to C₁₀₊ hydrocarbons or diesel fraction. The specific catalytic systems described herein make it possible to minimize the production of saturates and therefore maximize production of middle distillates, and optionally aromatics, which constitutes both an asset for the ethanol refiner and an advantage from the standpoint of lasting development.

Conversion of bio-oil(s) to the desired fuel product, or fuel product precursors (e.g., C₂-C₅ olefins) as in the case of C₁-C₅ alcohols, or mixtures thereof, in a single reactor configuration, can reduce processing costs. In one aspect, a process for converting one or more bio-oils to one or more C₂-C₅ olefins is provided. In some aspects, the process includes a single catalyst system (e.g., a doped zeolite), whereas in other aspects, the process includes a two or more catalyst system (e.g., a dehydration catalyst and a doped zeolite).

In one aspect, the process for producing one or more olefins can include: contacting an input stream with one or more catalysts in at least one reactor to form an output stream comprising the one or more olefins, in which the input stream includes one or more bio-oil(s) and the reactor is at a temperature from about 300° C. to about 600° C., a gauge pressure from about 1 to about 10 bar, and a weight hourly space velocity (WHSV) from about 0.5 h⁻¹ to about 10 h⁻¹. The input stream may further include a co-feed of water and/or one or more C₁-C₅ alcohols (e.g., methanol, ethanol, isobutanol, a mixture of at least C₄ and/or C₅ alcohols, or any combination thereof). The catalyst includes a zeolite and one or more dopants, where the one or more dopants can include one or more first metal dopants, one or more second metal dopants, or both. In certain aspects, the process can further include, prior to contacting the input stream, adding the one or more dopants to the catalyst.

In certain aspects, the one or more olefins in the output stream will include one or more first olefins. Non-limiting examples of first olefins include ethylene, propylene, butenes, and the like. In some aspects, the one or more first olefins can include the same olefin, and in other aspects, the one or more first olefins can include a mixture of different olefins. By way of example, in some aspects, the one or more first olefins can include a predominant first olefin, for example, propylene. As used herein, a “predominant first olefin” can be present at a greater weight percent than any other individual olefin in the one or more first olefins, for example, present in an amount that is at least about 20 weight percent, at least about 25 weight percent, or at least about 30 weight percent of the one or more first olefins. In some aspects, the predominant first olefin can be present in an amount of about 35 weight percent to about 40 weight percent of the one or more first olefins, in an amount of about 40 weight percent to about 45 weight percent of the one or more first olefins, in an amount of about 40 weight percent to about 50 weight percent of the one or more first olefins. It is further contemplated that the predominant first olefin can be present between any of these recited ranges.

The manufacture of zeolite types A, X, and Y is generally carried out by mixing and heating sodium aluminate and sodium silicate solutions, whereupon a sodium aluminosilicate gel is formed. The silicon oxide and aluminum oxide containing compounds pass into the liquid phase from which the zeolites are formed by crystallization. As such, the crude crystalline zeolite containing the original alkali metal may be subsequently converted to an intermediate ammonium form followed by calcination at 500° C.-550° C., to remove the ammonium counterion, thus yielding its final hydrogen form. In general, commercially produced hydrogen form zeolites (e.g., Clariant, Zeolyst, etc.) typically used for cracking, isomerizations, and alcohol to olefins chemistry have residual sodium levels of less than or equal to 0.05 wt % (e.g., 500 ppm). Higher residual levels of sodium (e.g., >2500 ppm) can severely deactivate the zeolite catalysts rendering them unacceptable for chemistries requiring highly acidic catalytic activity.

Non-limiting examples of suitable zeolites include crystalline silicates of the group ZSM-5 (MFI framework), BEA, CHA, FER, FAU, MWW, MOR, EUO, MFS, ZSM-48, MTT or TON having a SiO₂/AlO₃ ratio higher than 10, or a dealuminated crystalline silicate of the group ZSM5 (MFI or BEA frameworks), CHA, FER, FAU, MWW, MOR, EUO, MFS, ZSM-48, MTT or TON having a SiO₂/AlO₃ ratio higher than 10, and/or boron modified crystalline silicate of the group ZSM-5 (MFI or BEA frameworks), CHA, FER, FAU, MWW, MOR, EUO, MFS, ZSM-48, MTT or TON having a SiO₂/AlO₃ ratio higher than 10, or molecular sieves of the type silico-aluminophosphate of the group AEL. In some aspects, when the zeolite is a ZSM-5 zeolite, the ZSM-5 zeolite can have a SiO₂/AlO₃ ratio from about 23 to about 400. In certain aspects, the ZSM-5 zeolite can have a SiO₂/AlO₃ ratio from about 40 to about 300.

In some aspects, the zeolite can be doped with one or more dopants (also referred to as doped zeolite). In some aspects, the zeolite can be doped with one or more non-metal dopants (also referred to as non-metal doped zeolite). Non-limiting examples of non-metal dopants include boron, phosphor, germanium, among others. In one aspect, the one or more non-metal dopants only includes boron and phosphor.

In some aspects, the zeolite can be doped with only one dopant. In one aspect, the zeolite is only doped with boron. In another aspect, the zeolite is only doped with phosphor. In another aspect, the zeolite is only doped with both boron and phosphor.

In some aspects, the zeolite can be doped with one or more metal dopants (also referred to herein as a metal doped zeolite). Non-limiting examples of one or more metal dopants include sodium, potassium, lithium, beryllium, magnesium, calcium, strontium, barium, radium, or any combination thereof. In some aspects, the one or more metal dopants include one or more first metal dopants, one or more second metal dopants, or any combination thereof. In certain aspects, the one or more first metal dopants can include a Group 1A metal, such as sodium, lithium, potassium, or any combination thereof, and the one or second metal dopants can include a Group 2A metal, such as magnesium, calcium, strontium, or barium, or any combination thereof. In certain aspects, the metal dopant(s) may not only be from Group 1A or 2A, such as the non-limiting examples iron, tellurium, selenium, cobalt, nickel, lanthanum and/or other lanthanides, chromium, zirconium, ruthenium, molybdenum, iridium, tungsten, copper, manganese, vanadium, zinc, titanium, rhodium, rhenium, gallium, palladium, silver, indium, or any combination thereof.

In some aspects, the zeolite can be doped with only one metal dopant. In one aspect, the zeolite is only doped with sodium. In another aspect, the zeolite is only doped with lithium. In another aspect, the zeolite is only doped with potassium.

In certain aspects, the catalyst can include a zeolite doped with one or more first non-metal dopants and one or more metal dopants. In some aspects, the catalyst can include a zeolite doped with boron, phosphor, or both, and sodium, lithium, potassium, or any combination thereof. In one aspect, the catalyst can include a zeolite doped with sodium, boron, and phosphor. In other aspects, the catalyst can include a zeolite doped with lithium, boron, and phosphor. In yet other aspects, the catalyst can include a zeolite doped with potassium, boron and phosphor. In any of the foregoing aspects, the zeolite can be a ZSM-5 zeolite.

Boron and phosphor can be present in the catalysts in a variety of different amounts. In some aspects, the boron can be present in the catalyst in amount from about 0.5 wt. % to about 3 wt. %, including all the subranges in between. In certain aspects, the boron can be present in the catalyst in an amount from about 1 wt. % to about 3 wt. %, including all the subranges in between. In one aspect, the boron can be present in the catalyst in an amount of at least about 2 wt. %.

In some aspects, the phosphor can be present in the catalyst in amount from about 1 wt. % to about 5 wt. %, including all the subranges in between. In certain aspects, the phosphor can be present in the catalyst in an amount from about 2 wt. % to about 4 wt. %, including all the subranges in between. In one aspect, the phosphor can be present in the catalyst in an amount of at least about 3 wt. %.

In some aspects, the boron can be present in the catalyst in amount from about 0.5 wt. % to about 3 wt. %, and the phosphor can be present in the catalyst in an amount from about 1 wt. % to 5 wt. %. In certain aspects, the boron can be present in the catalyst in amount from about 1 wt. % to about 3 wt. %, and the phosphor can be present in the catalyst in an amount from about 2 wt. % to 4 wt. %. In one aspect, the boron can be present in the catalyst in amount of at least about 2 wt. %, and the phosphor can be present in the catalyst in an amount of at least about 3 wt. %.

In some aspects, the doped zeolite can be further doped with additional dopants, also referred to as other dopants, with or without the metal or non-metal dopants. In one aspect, the zeolite is doped with one or more non-metal dopants, one or more metal dopants, and one or more additional dopants. In another aspect, the zeolite is doped with one or more non-metal dopants and one or more other dopants. In another aspect, the zeolite is doped with one or more metal dopants and one or more other dopants. Non-limiting examples of additional dopants include iron, tellurium, selenium, cobalt, nickel, lanthanum and/or other lanthanides, chromium, zirconium, ruthenium, molybdenum, iridium, tungsten, copper, manganese, vanadium, zinc, titanium, rhodium, rhenium, gallium, palladium, silver, indium, or any combination thereof.

Granular or extruded catalyst(s) can be used for the reactions described herein. For example, in some aspects, granular or extruded catalyst(s) can have a particle size of greater than at least about 0.05 mm, about 0.1 mm or greater, or from about 0.05 mm to about 4.0 mm, including all the subranges in between. In one aspect, granular or extruded catalysts(s) can have a particle size from about 0.4 to about 2.5 mm.

In another aspect, two or more catalysts can be implemented for the conversion of bio-oil(s) to the desired fuel product, or fuel product precursors (e.g., C₂-C₅ olefins and/or aromatics) as in the case of bio-oil(s), or mixtures thereof, in a single reactor configuration. In some aspects, the process includes: contacting an input stream with at least a first catalyst and a second catalyst in a single reactor to form an output stream comprising the one or more first olefins (including a predominant first olefin) and optionally one or more aromatics, in which the input stream includes one or more bio-oil(s) and the single reactor is at a temperature from about 300° C. to about 600° C., a gauge pressure from 1 to about 10 bar, and a weight hourly space velocity (WHSV) from about 0.5 h⁻¹ to about 10 h⁻¹. The input stream may further include a co-feed of water and/or one or more C₁-C₅ alcohols (e.g., methanol, ethanol, isobutanol, a mixture of at least C₄ and/or C₅ alcohols, or any combination thereof). The first catalyst includes a zeolite and one or more dopants, in which the one or more dopants include one or more first dopants, one or more second dopants, or both. Furthermore, the two catalysts can be in a stacked bed configuration or admixed together. While a two or more catalyst system is described herein with respect to a single reactor configuration, it is also contemplated herein that such system can be implemented in a two-reactor configuration in which at least the first catalyst is in the first reactor and at least the second catalyst in the second reactor.

Exemplary catalyst combinations that can be used in the present two or more catalyst system and processes described herein, for example, physically mixed within a single-bed reactor, for olefin and/or aromatic formation, can include as a first part (e.g., a first catalyst) a doped zeolite, and as a second part (e.g., a second catalyst) can include the same or a different doped zeolite. In some aspects, the second catalyst includes a silicated, zirconated, titanated, niobium, or fluorinated [symbol] -alumina, an undoped [symbol] -alumina, a zeolite (undoped or doped), a silica alumina catalyst, a solid acid, or any combination thereof. In some aspects, the first catalyst can include a zeolite doped with boron, phosphor, or a combination thereof, and the second catalyst can include undoped gamma-alumina, zirconated [symbol] -alumina, or both. In one aspect, the first catalyst can include a zeolite doped with sodium, potassium, or lithium, or any combination thereof, and the second catalyst can include a zeolite doped with sodium, potassium, or lithium, or any combination thereof. In some aspects, the first catalyst can a zeolite doped with magnesium, calcium, strontium, barium, or any combination thereof, and the second catalyst can include undoped gamma-alumina, zirconated gamma-alumina, or both. In one aspect, the second catalyst can include a doped or undoped alumina catalyst. In one aspect, each of the two catalysts may be doped zeolites, but of different SiO₂/AlO₃ ratios or different groups, or each may contain different dopants, or each may contain different dopant loadings.

Non-limiting examples of suitable zeolites include crystalline silicates of the group ZSM-5 (MFI framework), BEA, CHA, FER, FAU, MWW, MOR, EUO, MFS, ZSM-48, MTT or TON having a SiO₂/AlO₃ ratio higher than 10, or a dealuminated crystalline silicate of the group ZSM5 (MFI or BEA frameworks), CHA, FER, FAU, MWW, MOR, EUO, MFS, ZSM-48, MTT or TON having a SiO₂/AlO₃ ratio higher than 10, and/or boron modified crystalline silicate of the group ZSM-5 (MFI or BEA frameworks), CHA, FER, FAU, MWW, MOR, EUO, MFS, ZSM-48, MTT or TON having a SiO₂/AlO₃ ratio higher than 10, or molecular sieves of the type silico-aluminophosphate of the group AEL. In some aspects, when the zeolite is a ZSM-5 zeolite, the ZSM-5 zeolite can have a SiO₂/AlO₃ ratio from about 23 to about 400. In certain aspects, the ZSM-5 zeolite can have a SiO₂/AlO₃ ratio from about 40 to about 300.

In some aspects, the zeolite can be doped with one or more dopants (also referred to as doped zeolite). In some aspects, the zeolite can be doped with one or more non-metal dopants (also referred to as non-metal doped zeolite). Non-limiting examples of non-metal dopants include boron, phosphor, germanium, among others. In one aspect, the one or more non-metal dopants only includes boron and phosphor.

In some aspects, the zeolite can be doped with only one dopant. In one aspect, the zeolite is only doped with boron. In another aspect, the zeolite is only doped with phosphor. In another aspect, the zeolite is only doped with both boron and phosphor.

In some aspects, the zeolite can be doped with one or more metal dopants (also referred to herein as a metal doped zeolite). Non-limiting examples of one or more metal dopants include sodium, potassium, lithium, beryllium, magnesium, calcium, strontium, barium, radium, or any combination thereof. In some aspects, the one or more metal dopants include one or more first metal dopants, one or more second metal dopants, or any combination thereof. In certain aspects, the one or more first metal dopants can include a Group 1A metal, such as sodium, lithium, potassium, or any combination thereof, and the one or second metal dopants can include a Group 2A metal, such as magnesium, calcium, strontium, or barium, or any combination thereof. In certain aspects, the metal dopant(s) may not only be from group 1A or 2A, such as the non-limiting examples iron, tellurium, selenium, cobalt, nickel, lanthanum and/or other lanthanides, chromium, zirconium, ruthenium, molybdenum, iridium, tungsten, copper, manganese, vanadium, zinc, titanium, rhodium, rhenium, gallium, palladium, silver, indium, or any combination thereof.

In some aspects, the zeolite can be doped with only one metal dopant. In one aspect, the zeolite is only doped with sodium. In another aspect, the zeolite is only doped with lithium. In another aspect, the zeolite is only doped with potassium.

In certain aspects, the catalyst can include a zeolite doped with one or more first non-metal dopants and one or more metal dopants. In some aspects, the catalyst can include a zeolite doped with boron, phosphor, or both, and sodium, lithium, potassium, or any combination thereof. In one aspect, the catalyst can include a zeolite doped with sodium, boron, and phosphor. In other aspects, the catalyst can include a zeolite doped with lithium, boron, and phosphor. In yet other aspects, the catalyst can include a zeolite doped with potassium, boron and phosphor. In any of the foregoing aspects, the zeolite can be a ZSM-5 zeolite.

Boron and phosphor can be present in the catalysts in a variety of different amounts. In some aspects, the boron can be present in the catalyst in amount from about 0.5 wt. % to about 3 wt. %, including all the subranges in between. In certain aspects, the boron can be present in the catalyst in an amount from about 1 wt. % to about 3 wt. %, including all the subranges in between. In one aspect, the boron can be present in the catalyst in an amount of at least about 2 wt. %.

In some aspects, the phosphor can be present in the catalyst in amount from about 1 wt. % to about 5 wt. %, including all the subranges in between. In certain aspects, the phosphor can be present in the catalyst in an amount from about 2 wt. % to about 4 wt. %, including all the subranges in between. In one aspect, the phosphor can be present in the catalyst in an amount of at least about 3 wt. %.

In some aspects, the boron can be present in the catalyst in amount from about 0.5 wt. % to about 3 wt. %, and the phosphor can be present in the catalyst in an amount from about 1 wt. % to 5 wt. %. In certain aspects, the boron can be present in the catalyst in amount from about 1 wt. % to about 3 wt. %, and the phosphor can be present in the catalyst in an amount from about 2 wt. % to 4 wt. %. In one aspect, the boron can be present in the catalyst in amount of at least about 2 wt. %, and the phosphor can be present in the catalyst in an amount of at least about 3 wt. %.

In some aspects, the doped zeolite can be further doped with additional dopants, also referred to as other dopants, with or without the metal or non-metal dopants. In one aspect, the zeolite is doped with one or more non-metal dopants, one or more metal dopants, and one or more additional dopants. In another aspect, the zeolite is doped with one or more non-metal dopants and one or more other dopants. In another aspect, the zeolite is doped with one or more metal dopants and one or more other dopants. Non-limiting examples of additional dopants include iron, tellurium, selenium, cobalt, nickel, lanthanum and/or other lanthanides, chromium, zirconium, ruthenium, molybdenum, iridium, tungsten, copper, manganese, vanadium, zinc, titanium, rhodium, rhenium, gallium, palladium, silver, indium, or any combination thereof.

Granular or extruded catalyst(s) can be used for the reactions described herein. For example, in some aspects, granular or extruded catalyst(s) can have a particle size of greater than at least about 0.05 mm, about 0.1 mm or greater, or from about 0.05 mm to about 4.0 mm, including all the subranges in between. In one aspect, granular or extruded catalysts(s) can have a particle size from about 0.4 to about 2.5 mm.

In certain aspects, the process includes: contacting an input stream with at least a first catalyst and a second catalyst in a single reactor to form an output stream that includes one or more first olefins (including a predominant first olefin) and, optionally one or more aromatics, in which the input stream includes one or more bio-oil(s) and the single bed reactor is at a temperature from about 300° C. to about 600° C., a gauge pressure from 1 to about 10 bar, and a weight hourly space velocity (WHSV) from about 0.5 h⁻¹ to about 10 h⁻¹, where the first catalyst includes a zeolite and one or more dopants; and the second catalyst includes a doped or undoped alumina catalyst including, in neutral or ionic form, one or more of zirconium (Zr), titanium (Ti), tungsten (W), fluorine (F), or silicon (Si). Furthermore, the two catalysts can be admixed together in the single bed reactor. The input stream may further include a co-feed of water and/or one or more C₁-C₅ alcohols (e.g., methanol, ethanol, isobutanol, a mixture of at least C₄ and/or C₅ alcohols, or any combination thereof).

In other aspects, a process for producing one or more olefins can include: contacting an input stream that includes the one or more bio-oil(s) with a first catalyst in a stacked bed reactor to form a first mixture that includes one or more first olefins (including a predominant first olefin) and optionally one or more aromatics. The stacked bed reactor is at a temperature from about 300° C. to about 600° C., a gauge pressure from 1 to about 10 bar, and a weight hourly space velocity (WHSV) from about 0.5 h⁻¹ to about 10 h⁻¹, and the first catalyst includes a doped or undoped zeolite. The input stream may further include a co-feed of water and/or one or more C₁-C₅ alcohols (e.g., methanol, ethanol, isobutanol, a mixture of at least C₄ and/or C₅ alcohols, or any combination thereof). The process further includes contacting the first mixture with at least a second catalyst, where the second catalyst includes an alumina catalyst including, in neutral or ionic form, one or more of zirconium (Zr), titanium (Ti), tungsten (W), fluorine (F), or silicon (Si) to produce the output stream that includes one or more olefins (e.g., C₂-C₅ olefins) and/or aromatics. In such instances, the first catalyst can be impregnated into a first catalyst bed of the stacked bed reactor, and the second catalyst can be impregnated into a second catalyst bed of the stacked bed reactor.

Regarding the output stream, the first olefins (e.g., C₂-C₅ olefins) can be present in an amount that is at least 60 wt. % of the total amount of unsaturated hydrocarbons present in the vapor phase of the output stream, excluding CO, CO₂ and hydrogen. The first olefins can be present in an amount that is at least 70 wt. % of the total amount of unsaturated hydrocarbons present in the output stream. The first olefins can be present in an amount that is at least 80 wt. % of the total amount of unsaturated hydrocarbons present in the output stream. The first olefins can be present in an amount that is at least 90 wt. % of the total amount of unsaturated hydrocarbons present in the output stream. The first olefins can be present in an amount from about 65 wt. % to about 95 wt. %, from about 65 wt. % to about 85 wt. %, or from about 70 wt. % to about 80 wt. %, including all the subranges in between, of the total amount of unsaturated hydrocarbons present in the output stream. The one or more aromatics can be present in an amount from about 65 wt. % to about 80 wt. %, or from about 75 wt. % to about 80 wt. %, including all the subranges in between, of the total amount of unsaturated hydrocarbons present in the output stream. Further regarding the output stream, the processes disclosed herein can further include removing at least a portion of the C₂ olefins from the output stream. The processes can include removing at least a portion of the C₄ olefins from the output stream. The processes can include removing at least a portion of the C₅ olefins from the output stream. The processes can include removing at least a portion of the one or more aromatics from the output stream.

In certain aspects, the one or more olefins in the output stream will include one or more first olefins. Non-limiting examples of first olefins include ethylene, propylene, butenes, and the like. In some aspects, the one or more first olefins can include the same olefin, and in other aspects, the one or more first olefins can include a mixture of different olefins. By way of example, in some aspects, the one or more first olefins can include a predominant first olefin, for example, propylene. As used herein, a “predominant first olefin” can be present at a greater weight percent than any other individual olefin in the one or more first olefins, for example, present in an amount that is at least about 20 wt. %, at least about 25 wt. %, or at least about 30 wt. % of the one or more first olefins. In some aspects, the predominant first olefin can be present in an amount of about 35 wt. % to about 40 wt. % of the one or more first olefins, in an amount of about 40 wt. % to about 45 wt. % of the one or more first olefins, in an amount of about 40 wt. % to about 50 wt. % of the one or more first olefins. It is further contemplated that the predominant first olefin can be present between any of these recited ranges.

In some aspects, one or more unsaturated hydrocarbons present in the output stream can include one or more low carbon intensity unsaturated hydrocarbons. In one aspect, all the unsaturated hydrocarbons can be low carbon intensity unsaturated hydrocarbons. As used herein, low carbon intensity when used to modify an unsaturated hydrocarbon (e.g., one or more unsaturated hydrocarbons) refers to a carbon intensity that is at least about 50% less than a typical carbon intensity for its petroleum equivalent.

In some aspects, one or more unsaturated hydrocarbons present in the output stream can include one or more zero carbon intensity hydrocarbons. In one aspect, all the unsaturated hydrocarbons can be zero carbon intensity hydrocarbons. As used herein, zero carbon intensity when used to modify an unsaturated hydrocarbon (e.g., one or more unsaturated hydrocarbons) refers to a carbon intensity that is at least about 90% to 100% less than a typical carbon intensity for its petroleum equivalent.

In some aspects, one or more unsaturated hydrocarbons present in the output stream can include one or more negative carbon intensity hydrocarbons. In one aspect, all the one or more hydrocarbons can be negative carbon intensity hydrocarbons. As used herein, negative carbon intensity when used to modify an unsaturated hydrocarbon refers to a carbon intensity that is more than 100% less than a typical carbon intensity for its petroleum equivalent.

In some aspects, the output stream includes saturates. In certain aspects, a total amount of saturates present in the output stream does exceed about 20 wt. %. In one aspect, a total amount of saturates present in the output stream does not exceed about 15 wt. %. In other aspects, the total amount of saturates present in the output stream can be from about 5 wt. % to about 15 wt. % or from about 5 wt. % to about 10 wt. %, including all subranges in between.

Regarding the reactor, the reactor can be operated at a temperature from about 300° C. to about 600° C., including all the subranges in between. The reactor can be operated at a temperature from about 350° C. to about 500° C., including all the subranges in between. The reactor can be operated at a temperature from about 450° C. to about 500° C., including all the subranges in between. The reactor can be operated at a gauge pressure from about 1 to about 10 bar, including all the subranges in between. The reactor can be operated at a gauge pressure from 1 to about 5 bar, including all the subranges in between. The reactor can be operated at a gauge pressure from 1 to about 2 bar, including all the subranges in between. The reactor can be operated at a gauge pressure of about 5 or lower. The reactor can be operated at a WHSV from about 0.5 h⁻¹ to about 10 h⁻¹, including all the subranges in between. The reactor can be operated at a WHSV from about 1.0 h⁻¹ to about 5.0 h⁻¹, from about 2.0 h⁻¹ to about 5.0 h⁻¹, from about 3.0 h⁻¹ to about 5.0 h⁻¹, or from about 5.0 h⁻¹ to about 10 h⁻¹, including all the subranges in between. The reactor can be a fixed bed reactor. The reactor can be a fluidized bed reactor. The reactor can be a moving bed reactor.

The disclosure also describes a process for converting one or more bio-oil(s) to one or more first olefins (including a predominant first olefin) and/or one or more aromatics using a single catalyst system. The use of a single catalyst system can be desirable in a variety of instances, for example, when some portion of unconverted bio-oil(s) and related oxygenates are acceptable in the output stream, or when the catalyst is continuously regenerated during operation, which can be implemented in, for example, fluidized bed or moving bed reactors. For the purposes of this disclosure, an “oxygenate” is a hydrocarbon that contains oxygen as part of its chemical structure. In some aspects, the one or more bio-oil(s) in an input stream of the single can be one or more edible or non-edible (e.g., used) vegetable oils, such as corn oil, soybean oil, olive oil, peanut oil, rapeseed oil, including canola, coconut oil, palm oil, or animal based products such as tallow or lard, among others. In some aspects, the bio-oils, fatty acids/esters, or mixtures thereof may be subjected to partial or complete hydrogenation, and/or the olefin isomerized prior to use as feed. In some aspects, the bio-oils include fatty acids/esters are derived from triglycerides, for example, by hydrolysis of bio-oils to fatty acids or transesterification with alcohols to fatty acid esters. In some aspects, the bio-oils, fatty acids/esters, or mixtures thereof can be produced by fermentative processes. In some aspects, the input stream may further include a co-feed of water and/or one or more C₁-C₅ alcohols (e.g., methanol, ethanol, isobutanol, a mixture of at least C₄ and/or C₅ alcohols, or any combination thereof).

Conversion of bio-oil(s) to the desired fuel product, or fuel product precursors (e.g., C₂-C₅ olefins and/or aromatics) as in the case of bio-oil(s), or mixtures thereof with a single catalyst system can, for example, reduce processing costs and simplify and optimize the conversion process that would not otherwise be possible with a two-catalyst system. In these processes, the single catalyst system includes only one catalyst, such as zeolite. In some aspects, the only one catalyst is not a doped or undoped alumina catalyst. In certain aspects, the zeolite can be a zeolite doped with one or more non-metal dopants (e.g., boron, phosphor, both), and optionally, further doped with an additional dopant.

In one aspect, the one catalyst can include a zeolite and one or more dopants, in which the one or more dopants include one or more first dopants (e.g., boron, phosphor, or both). In another aspect, the one catalyst can include a zeolite and one or more first dopants, in which the one or more first metal dopants include boron, phosphor, or both, and optionally one or more dopants.

Alternatively, or in addition, the one or more dopants can include one or more second dopants (e.g., beryllium, magnesium, calcium, strontium, barium, radium, or any combination thereof). In one aspect, one catalyst can include a zeolite and one or more first non-metal dopants and one or more second metal dopants, in which the one or more second metal dopants include calcium, magnesium, strontium, or any combination thereof. In another aspect, the one catalyst can include a zeolite and one or more second metal dopants, in which the one or more second metal dopants include magnesium, calcium, strontium, or barium, or any combination thereof, and one or more non-metal dopants, in which the one or more non-metal dopants include boron, phosphor, or a combination thereof.

Non-limited examples of zeolites olefin formation can include doped zeolites such as crystalline silicates of the group ZSM-5 (MFI or BEA frameworks), CHA, FER, FAU, MWW, MOR, EUO, MFS, ZSM-48, MTT or TON having a SiO₂/AlO₃ ratio higher than 10, and/or boron modified crystalline silicate of the group ZSM-5 (MFI or BEA frameworks), CHA, FER, FAU, MWW, MOR, EUO, MFS, ZSM-48, MTT or TON having a SiO₂/AlO₃ ratio higher than 10, or a dealuminated crystalline silicate of the group ZSM5 (MFI or BEA frameworks), CHA, FER, FAU, MWW, MOR, EUO, MFS, ZSM-48, MTT or TON having a SiO₂/AlO₃ ratio higher than 10. In some aspects, when the zeolite is a ZSM-5 zeolite, the ZSM-5 zeolite can have a SiO₂/AlO₃ ratio from about 23 to about 400. In certain aspects, the ZSM-5 zeolite can have a SiO₂/AlO₃ ratio from about 40 to about 300.

In some aspects, the zeolite can be doped with one or more metal dopants (also referred to herein as a metal doped zeolite). Non-limiting examples of one or more metal dopants include sodium, potassium, lithium, beryllium, magnesium, calcium, strontium, barium, radium, or any combination thereof. In some aspects, the one or more metal dopants include one or more first metal dopants, one or more second metal dopants, or any combination thereof. In certain aspects, the one or more first metal dopants can include a Group 1A metal, such as sodium, lithium, potassium, or any combination thereof, and the one or second metal dopants can include a Group 2A metal, such as magnesium, calcium, strontium, or barium, or any combination thereof. In certain aspects, the metal dopant(s) may not only be from group 1A or 2A, such as the non-limiting examples iron, tellurium, selenium, cobalt, nickel, lanthanum and/or other lanthanides, chromium, zirconium, ruthenium, molybdenum, iridium, tungsten, copper, manganese, vanadium, zinc, titanium, rhodium, rhenium, gallium, palladium, silver, indium, or any combination thereof.

In some aspects, the zeolite can be doped with one or more non-metal dopants. A zeolite doped with one or more non-metal dopants is also referred to herein as a non-metal doped zeolite. Non-limiting examples of non-metal dopants include boron, phosphor, germanium, among others. In one aspect, the one or more non-metal dopants only includes boron and phosphor.

In some aspects, the zeolite can be doped with only one dopant. In one aspect, the zeolite is only doped with boron. In another aspect, the zeolite is only doped with phosphor. In another aspect, the zeolite is only doped with both boron and phosphor.

In some aspects, the zeolite can be doped with only one metal dopant. In one aspect, the zeolite is only doped with sodium. In another aspect, the zeolite is only doped with lithium. In another aspect, the zeolite is only doped with potassium.

In certain aspects, the catalyst can include a zeolite doped with one or more first non-metal dopants and one or more metal dopants. In some aspects, the catalyst can include a zeolite doped with boron, phosphor, or both, and sodium, lithium, potassium, or any combination thereof. In one aspect, the catalyst can include a zeolite doped with sodium, boron, and phosphor. In other aspects, the catalyst can include a zeolite doped with lithium, boron, and phosphor. In yet other aspects, the catalyst can include a zeolite doped with potassium, boron and phosphor. In any of the foregoing aspects, the zeolite can be a ZSM-5 zeolite.

Boron and phosphor can be present in the catalysts in a variety of different amounts. In some aspects, the boron can be present in the catalyst in amount from about 0.5 wt. % to about 3 wt. %, including all the subranges in between. In certain aspects, the boron can be present in the catalyst in an amount from about 1 wt. % to about 3 wt. %, including all the subranges in between. In one aspect, the boron can be present in the catalyst in an amount of at least about 2 wt. %.

In some aspects, the phosphor can be present in the catalyst in amount from about 1 wt. % to about 5 wt. %, including all the subranges in between. In certain aspects, the phosphor can be present in the catalyst in an amount from about 2 wt. % to about 4 wt. %, including all the subranges in between. In one aspect, the phosphor can be present in the catalyst in an amount of at least about 3 wt. %.

In some aspects, the boron can be present in the catalyst in amount from about 0.5 wt. % to about 3 wt. %, and the phosphor can be present in the catalyst in an amount from about 1 wt. % to 5 wt. %. In certain aspects, the boron can be present in the catalyst in amount from about 1 wt. % to about 3 wt. %, and the phosphor can be present in the catalyst in an amount from about 2 wt. % to 4 wt. %. In one aspect, the boron can be present in the catalyst in amount of at least about 2 wt. %, and the phosphor can be present in the catalyst in an amount of at least about 3 wt. %.

In one exemplary aspect, a process for producing one or more olefins using a single catalyst system can include contacting an input stream that includes the one or more bio-oil(s) with a catalyst in a reactor to form an output stream that includes the one or more first olefins (including a predominant olefin) and, optionally, one or more aromatics, in which the catalyst consists essentially of zeolite doped with boron and phosphor and, optionally, one or more additional dopants. The input stream may further include a co-feed of water and/or one or more C₁-C₅ alcohols (e.g., methanol, ethanol, isobutanol, a mixture of at least C₄ and/or C₅ alcohols, or any combination thereof). The reactor is at a temperature from about 300° C. to about 600° C., a gauge pressure from 1 to about 10 bar, and a weight hourly space velocity (WHSV) from about 0.5 h⁻¹ to about 10 h⁻¹.

In some aspects, the process can include, after contacting the input stream with the catalyst in the reactor, regenerating the catalyst. In some aspects, the regeneration of the catalyst can be carried out by purging any gaseous or liquid hydrocarbons or oxygenates from the reactor and then introducing air and/or oxygen optionally diluted with inert gas or steam to combust any solid carbon deposits on the catalyst. In some aspects, the process can include, a system whereby the catalyst is circulated between a reactor in which it is contacted with the input stream and a regeneration reactor in which is it contacted with air and/or oxygen optionally diluted with inert gas or steam to combust any solid carbon deposits on the catalyst.

In some aspects, the process can further include contacting another input stream that includes the one or bio-oil(s), and optionally the co-feed of water and/or one or more C₁-C₅ alcohols (e.g., methanol, ethanol, isobutanol, a mixture of at least C₄ and/or C₅ alcohols, or any combination thereof), with the regenerated catalyst (e.g., the catalyst post-regeneration) in the reactor to form another output stream comprising one or more C₂-C₅ olefins and/or aromatics. A person skilled in the art will appreciate that the regenerated catalyst can have a lower concentration of boron, phosphor, or both compared to the catalyst prior to regeneration.

Regarding the reactor, the reactor can be operated at a temperature from about 300° C. to about 600° C., including all the subranges in between. The reactor can be operated at a temperature from about 350° C. to about 500° C., including all the subranges in between. The reactor can be operated at a temperature from about 450° C. to about 500° C., including all the subranges in between. The reactor can be operated at a gauge pressure from about 1 to about 10 bar, including all the subranges in between. The reactor can be operated at a gauge pressure from 1 to about 5 bar, including all the subranges in between. The reactor can be operated at a gauge pressure from 1 to about 2 bar, including all the subranges in between. The reactor can be operated at a gauge pressure of about 5 or lower. The reactor can be operated at a WHSV from about 0.5 h⁻¹ to about 10 h⁻¹, including all the subranges in between. The reactor can be operated at a WHSV from about 1.0 h⁻¹ to about 5.0 h⁻¹, from about 2.0 h⁻¹ to about 5.0 h⁻¹, from about 3.0 h⁻¹ to about 5.0 h⁻¹, or from about 5.0 h⁻¹ to about 10 h⁻¹, including all the subranges in between. The reactor can be a fixed bed reactor. The reactor can be a fluidized bed reactor. The reactor can be a moving bed reactor.

Further regarding the output stream, the processes disclosed herein can further include removing at least a portion of the predominant olefins. The processes can include removing at least a portion of the C₂ olefins from the output stream. The processes can include removing at least a portion of the C₃ olefins from the output stream. The processes can include removing at least a portion of the C₄ olefins from the output stream. The processes can include removing at least a portion of the C₅ olefins from the output stream. The processes can include removing at least a portion of the one or more aromatics from the output stream.

In one exemplary aspect, a process for producing one or more olefins using a two-catalyst system can include contacting an input stream in at least one reactor with at least a first catalyst and a second catalyst to form an output stream comprising the one or more olefins, the input stream comprising one or more bio-oils, the first catalyst comprises a zeolite and two dopants, and the second catalyst comprises a doped alumina catalyst. The input stream may further include a co-feed of water and/or one or more C₁-C₅ alcohols (e.g., methanol, ethanol, isobutanol, a mixture of at least C₄ and/or C₅ alcohols, or any combination thereof). The output stream includes one or more first olefins (including a predominant olefin), and optionally, one or more aromatics. The first catalyst consists of a zeolite (e.g., a ZSM-5 zeolite) doped with boron and/or phosphor and optionally one or more additional dopants. The second catalyst consists of a doped alumina catalyst, such as a silicated [symbol] -alumina, zirconated [symbol] -alumina, titanated [symbol] -alumina, niobium [symbol] -alumina, or fluorinated [symbol] -alumina, an undoped [symbol] -alumina, an undoped zeolite, a silica alumina catalyst, or any combination thereof. The reactor is at a temperature from about 450° C. to about 500° C., a gauge pressure from about 1 to about 2 bar, and a weight hourly space velocity (WHSV) from about 2.0 h⁻¹ to about 5.0 h⁻¹. The boron is present in the first catalyst in an amount of about 2 wt. % and the phosphor is present in the catalyst in an amount of about 3 wt. %. The zeolite may be a ZSM-5 zeolite.

FIG. 2 shows an exemplary reactor system 1000. As shown, an input 100, such as one or more bio-oil(s), optionally with a co-feed of inert water and/or one or more C₁-C₅ alcohols (e.g., methanol, ethanol, isobutanol, a mixture of at least C₄ alcohols, a mixture of at least C₅ alcohols, or a mixture of at least C₄ and C₅ alcohols), can be fed into a reactor 300, to produce an output 200, such as an olefin mixture that includes a predominant olefin and optionally one or more aromatics. Additionally, recycle streams R1, R2, and R3 may recycle C₂, C₄, and C₅ olefins respectively, back into the input 100 to be fed back into the reactor 300. Water 400 as a biproduct can be a result of any water co-fed into reactor 300 and may also be produced in situ by the reactor 300, via dehydration of ethanol to ethylene, and thus condensed and removed and/or a portion recycled back into the input 100 as an inert co-feed back into the reactor 300 (not shown) as part of the output 200.

Reactor 300 can have a variety of configurations. In some aspects, the reactor is a single bed reactor (e.g., a single fixed bed reactor, single fluidized bed reactor, single moving bed reactor). In such aspects, the single catalyst bed (not shown) of the reactor 300 can be impregnated with two or more catalysts so as to form a mixed catalyst bed.

In other aspects, the reactor 300 can include two or more catalyst beds (e.g., a stacked configuration). In such aspects, a first catalyst bed can include one or more catalysts impregnated therein and the second catalyst bed can include one or more catalysts impregnated therein. For example, the first catalyst bed can include a first catalyst that includes a zeolite doped with at least one or more dopants (e.g., boron, phosphor, or both); and the second catalyst bed can include a second catalyst that includes a silicated [symbol] -alumina, zirconated [symbol] -alumina, titanated [symbol] -alumina, niobium [symbol] -alumina, or fluorinated [symbol] -alumina, an undoped [symbol] -alumina, a zeolite (undoped or doped), a silica alumina catalyst, a solid acid, or any combination thereof. The doped zeolite of the first catalyst can be further doped with one or more metal or non-metal dopants. Alternatively, or in addition, the doped zeolite can be doped with at least one or more other dopants. In other aspects, the first catalyst bed can include a mixture of the first catalyst and the second catalyst impregnated therein.

In some aspects, the first catalyst bed does not contain the second catalyst. Alternatively, or in addition, the second catalyst bed does not contain the first catalyst.

In other aspects, the reactor system can include two or more reactors in series. In such aspects, for example, when there is a first reactor and a second reactor, the first reactor, the second reactor, or both can have any reactor configuration (e.g., structural design, such as single bed, mixed bed, stacked bed, and the like) disclosed herein. In some aspects, the first reactor and the second reactor have the same configuration (e.g., structural design). In other aspects, the first reactor and the second reactor have different configurations (e.g., structural design). Alternatively, or in addition, the first reactor and the second reactor can operate at the same process conditions (e.g., temperature, pressure, WHSV, and the like). In other aspects, the first reactor and the second reactor can operate at different process conditions. Alternatively, or in addition, the first reactor and the second reactor can each have a catalyst bed and both beds can be impregnated with the same catalyst(s). In other aspects, the catalyst bed(s) of the first reactor can be impregnated with one or more first catalysts and the catalyst bed(s) of the second reactor can be impregnated with one or more second catalyst that are different that the first catalysts.

The following specific examples are intended to be illustrative of the invention and should not be construed as limiting the scope of the invention as defined by appended claims.

### EXAMPLES

### Example 1: Reactor Set-Up

The conversion of bio-oils, fatty acids/esters, or mixtures thereof, optionally combined with a co-feed of C₁-C₅ alcohols and/or water, to C₂-C₅ olefins and aromatics was carried out at 300° C.-600° C., via fixed bed reactors, containing specified catalyst(s), and flowing preheated (180° C.) bio-oils, fatty acids/esters, or mixtures thereof, with or without the co-feed of C₁-C₅ alcohols and/or water, in a downward flow over the fixed catalyst bed while co-feeding nitrogen at atmospheric pressure or under moderate pressures (i.e., 0-30 bar). The flow rate of bio-oils, fatty acids/esters, or mixtures thereof, optionally with the co-feed of C₁-C₅ alcohols and/or water, was controlled by Teledyne Model 500D syringe pumps, and the flow rates were adjusted to obtain the targeted olefin WHSV (weight hourly space velocity). The internal reaction temperature was maintained constant via a Lindberg Blue M furnace as manufactured by Thermo-Scientific. The conversion and selectivity of bio-oils, fatty acids/esters, or mixtures thereof was calculated by analysis of the liquid phase reactor effluent by GC for organic and water content, online GC analysis of non-condensed hydrocarbons (i.e., C₂-C₅ olefins), and on-line thermal conductivity detector for quantitation of CO, CO₂ and relative to nitrogen as internal standard. Thus, passing a vaporized stream of corn oil over the catalyst combination in a single fixed bed reactor at between 460° C.-500° C. results in the formation of C₂-C₅ olefins and aromatics in high yields.

### Example 2: Preparation of Catalysts

Boron and/or phosphor impregnated ZSM-5 zeolite catalyst preparation: boron and phosphor impregnated zeolite catalyst was prepared by incipient wetness technique as described. 0.83 g phosphoric acid (85%) and 0.94 g boric acid was dissolved in deionized water (7.2 ml). Upon dissolution, the solution was added in dropwise fashion to 6 g ZSM-5 zeolite support (i.e., Clariant H-CZP-90E). The resulting impregnated catalyst was dried at 160° C. for 1 hr, and afterwards calcined at 550° C. for 3 hrs.

Impregnated Zr- [symbol] -Alumina (nominal Zr metal 5 wt. %) Catalyst preparation: Zr- [symbol] -alumina catalyst was prepared by incipient wetness technique as described. The precursor metal salts (Sigma Aldrich): 2.64 g zirconium (IV) oxynitrate hydrate was dissolved in deionized water (14.9 ml). Upon salt dissolution, the solution was added in dropwise fashion to 15 g [symbol] -alumina support. The resulting mixed metal oxide was manually mixed to assure complete wetting, and the resulting impregnated catalyst was dried at 160° C. for 1 hr, and afterwards calcined at 500° C. for 4 hrs.

### Example 3: Single Stage Reactor—Food Grade Corn Oil Feed

Single Stage reactor configuration: Reaction Conditions: Feed pre-heater=180° C., Reactor T=460° C. in reactor, Corn Oil flow=0.30 ml/min, WHSV=3.35 (corn oil basis), Nitrogen=10 ml/min, P=0 bar; Catalysts—Zirconated (4.0 wt %) [symbol] -Alumina physically mixed with doped ZSM-5 zeolite.

Single pass reactor effluent composition and corresponding weight percent of total (Table 2A shows the vapor phase and Table 2B shows the liquid phase).

TABLE 2A

| Component | Gas Phase Wt % (represents 54% of converted bio-oil) |
|---|---|
| Methane | 0.99 |
| Ethylene | 7.83 |
| Ethane | 1.66 |
| Propylene | 23.06 |
| Propane | 6.35 |
| isobutane | 5.64 |
| isobutylene | 7.36 |
| n-butane | 3.34 |
| n-butene | 4.91 |
| trans-2-butene | 4.57 |
| cis-2-butene | 3.16 |
| pentenes | 5.42 |
| pentanes | 2.02 |
| CO₂ | 11.67 |
| CO | 6.85 |
| Hydrogen | 4.95 |
| Total Saturates | 19.01 |

TABLE 2B

| Organic Phase | Area % of Organic Phase |
|---|---|
| Analysis | (represents 46% of converted bio-oil) |
| C4's | 4.52 |
| C5's | 5.35 |
| C6's | 4.97 |
| Benzene | 7.46 |
| toluene | 22.3 |
| ethyl benzene | 4.36 |
| p/m-xylene | 18.78 |
| o-xylene | 5.25 |
| C9 Aromatics | 9.19 |
| C10+ | 14.62 |

As shown, the vapor phase olefin content was about 5800 and the aromatic content in the liquid phase was about 6700.

### Example 4: Single Stage Reactor—Food Grade Corn Oil+DI Water Feed

Single Stage reactor configuration: Reaction Conditions: Feed pre-heater=180° C., Reactor T=480° C. in reactor, Corn Oil flow=0.20 ml/min, deionized (DI) Water Flow=0.10 ml/min; WHSV=4.43 (corn oil basis), Nitrogen=10 ml/min, P=0 bar; Catalysts—Boron/Phosphor doped ZSM-5 zeolite (SiO₂/AlO₃ ratio=90).

Single pass reactor effluent composition and corresponding weight percent of total as a percent of corn oil mass fed (Table 3A shows the vapor phase and Table 3B shows the liquid phase).

TABLE 3A
(Vapor phase = 33% of Corn Oil mass
fed is converted), CO₂/CO not measured

| Component | Vapor GC Area % |
|---|---|
| ethylene | 9.7 |
| methane | 0.54 |
| ethane | 0.86 |
| propylene | 32.07 |
| propane | 2.46 |
| isobutane | 2.08 |
| butane | 1.22 |
| butadiene | 0.16 |
| trans-2-butene | 7.47 |
| cis-2-butene | 5.44 |
| n-butene + (isobutylene) iC4 | 17.59 |
| 2-pentene (mixed isomers) | 4.66 |
| 1-pentene | 0.8 |
| pentane | 0.47 |
| 3-methyl-1-butene (3M1B) | 0.73 |
| 2-methyl-1-butene (2M1B) | 5.12 |
| 2-methyl-2-butene (2M2B) | 7.14 |
| isopentane | 1.08 |
| Total Saturates | 8.17 |

TABLE 3B
(Liquid phase = 67% of Corn Oil mass fed is converted)

| Carbon Number | GC Area % - Liquid Composition |
|---|---|
| C2 | 0 |
| C3 | 0.64 |
| C4 | 3.04 |
| C5-C7 | 18.5 (28% benzene) |
| C8 | 19.6 (70% toluene) |
| C9-C11 | 28.8 (75% xylenes, 25% trimethylbenzenes) |
| C12 | 16.1 (100% naphthalenes) |
| C13-C15 | 1.8 |
| C16 | 0.92 |
| C20+ | 10.65 (corn oil starting material) |

As shown, the vapor phase olefin content was about 750 and the aromatic content in the liquid phase was about 650.

### Example 5: Single Stage Reactor—Food Grade Corn Oil+EtOH+DI Water Feed

Single Stage reactor configuration: Reaction Conditions: Feed pre-heater=180° C., Reactor T=480° C. in reactor, Corn Oil flow=0.10 ml/min, ethanol (EtOH)/DJ Water (50/50) Flow=0.20 ml/min; WHSV=4.35 (corn oil+EtOH basis), Nitrogen=10 ml/min, P=0 bar; Catalysts—Boron/Phosphor doped ZSM-5 zeolite (SiO₂/AlO₃ ratio=90).

Single pass reactor effluent composition and corresponding weight percent of total as a percent of corn oil mass fed (Table 4A shows the vapor phase and Table 4B shows the liquid phase).

TABLE 4A
(Vapor phase = 46% of Corn Oil + EtOH
mass fed), trace levels of CO₂/CO detected

| Component | Vapor GC Area % |
|---|---|
| ethylene | 38.78 |
| methane | 0.18 |
| ethane | 0.61 |
| propylene | 26.58 |
| propane | 1.84 |
| isobutane | 1.77 |
| butane | 0.8 |
| butadiene | 0 |
| trans-2-butene | 4.6 |
| cis-2-butene | 3.44 |
| n-butene + (isobutylene) iC4 | 11.48 |
| 2-pentene (mixed isomers) | 2.18 |
| 1-pentene | 0.5 |
| pentane | 0.2 |
| 3-methyl-1-butene (3M1B) | 0.34 |
| 2-methyl-1-butene (2M1B) | 1.83 |
| 2-methyl-2-butene (2M2B) | 4.03 |
| isopentane | 0.84 |
| Total Saturates | 6.06 |

TABLE 4B
(Liquid phase = 54% of Corn Oil + EtOH mass fed)

| Carbon Number | GC Area % - Liquid Composition |
|---|---|
| C2 | 0 |
| C3 | 0.52 |
| C4 | 1.9 |
| C5-C7 | 15.5 (38% benzene) |
| C8 | 23.4 (78% toluene) |
| C9-C11 | 36.4 (75% xylenes, 25% trimethylbenzenes) |
| C12 | 18.13 (100% naphthalenes) |
| C13-C15 | 2.17 |
| C16 | 1.04 |
| C20+ | 0.99 (corn oil) |

As shown, the vapor phase olefin content was about 80% and the aromatic content in the liquid phase was about 750.

### Example 6: Single Stage Reactor (Food Grade Corn Oil+EtOH+DI Water Feed)

Single Stage reactor configuration: Reaction Conditions: Feed pre-heater=180° C., Reactor T=485° C. in reactor, Corn Oil flow=0.03 ml/min, EtOH/I Water (50/50) Flow=0.12 ml/min; WHSV=2.29 (corn oil+EtOH basis), Nitrogen=10 ml/min, P=0 bar; Catalysts—Boron/Phosphor doped ZSM-5 zeolite (SiO₂/AlO₃ ratio=90).

Single pass reactor effluent composition and corresponding weight percent of total as a percent of corn oil mass fed (Table 5A shows the vapor phase and Table 5B shows the liquid phase).

TABLE 5A
(Vapor phase = 43% of Corn Oil +
EtOH mass fed), CO₂/CO not measured

| Component | Vapor GC Area % |
|---|---|
| ethylene | 45.73 |
| methane | 0.23 |
| ethane | 0.8 |
| propylene | 24.97 |
| propane | 2.28 |
| isobutane | 2.08 |
| butane | 0.75 |
| butadiene | 0.03 |
| trans-2-butene | 3.53 |
| cis-2-butene | 2.62 |
| n-butene + (isobutylene) iC4 | 9.49 |
| 2-pentene (mixed isomers) | 1.51 |
| 1-pentene | 0.36 |
| pentane | 0.21 |
| 3-methyl-1-butene (3M1B) | 0.27 |
| 2-methyl-1-butene (2M1B) | 1.65 |
| 2-methyl-2-butene (2M2B) | 2.8 |
| isopentane | 0.7 |
| Total Saturates | 6.82 |

TABLE 5B
(Liquid phase = 57% of Corn Oil + EtOH mass fed)

| Carbon Number | GC Area % - Liquid Composition |
|---|---|
| C2 | 0 |
| C3 | 0.1 |
| C4 | 1.16 |
| C5-C7 | 14.3 (35% benzene) |
| C8 | 24.4 (83% toluene) |
| C9-C11 | 40.2 (78% xylenes, 22% trimethylbenzenes) |
| C12 | 16.32 (100% naphthalenes) |
| C13-C15 | 2.27 |
| C16 | 1.02 |
| C20+ | 0.25 (corn oil) |

As shown, the vapor phase olefin content was about 800% and the aromatic content in the liquid phase was about 8000.

### Example 7: Single Stage Reactor—Food Grade Corn Oil+EtOH Feed

Single Stage reactor configuration: Reaction Conditions: Feed pre-heater=180° C., Reactor T=480° C. in reactor, Corn Oil flow=0.10 ml/min, EtOH (100) Flow=0.12 ml/min; WHSV=4.52 (corn oil+EtOH basis), Nitrogen=10 ml/min, P=0 bar; Catalysts—Boron/Phosphor doped ZSM-5 zeolite (SiO₂/AlO₃ ratio=90).

Single pass reactor effluent composition (vapor phase ‘Table 6A’ and liquid phase ‘Table 6B’) and corresponding weight percent of total as a percent of corn oil mass fed.

TABLE 6A
(Vapor phase = 54% of Corn Oil +
EtOH mass fed), CO₂/CO not measured

| Component | Vapor GC Area % |
|---|---|
| ethylene | 37.61 |
| methane | 0.51 |
| ethane | 1.49 |
| propylene | 27.77 |
| propane | 2.31 |
| isobutane | 1.86 |
| butane | 0.93 |
| butadiene | 0.03 |
| trans-2-butene | 4.45 |
| cis-2-butene | 3.27 |
| n-butene + (isobutylene) iC4 | 11.87 |
| 2-pentene (mixed isomers) | 1.78 |
| 1-pentene | 0.4 |
| pentane | 0.31 |
| 3-methyl-1-butene (3M1B) | 0.33 |
| 2-methyl-1-butene (2M1B) | 1.61 |
| 2-methyl-2-butene (2M2B) | 2.89 |
| isopentane | 0.63 |
| Total Saturates | 7.53 |

TABLE 6B
(Liquid phase = 46% of Corn Oil + EtOH mass fed)

| Carbon Number | GC Area % - Liquid Composition |
|---|---|
| C2 | 0 |
| C3 | 0.59 |
| C4 | 2.9 |
| C5-C7 | 16.7 (30% benzene) |
| C8 | 21.3 (75% toluene) |
| C9-C11 | 34.7 (73% xylenes, 27% trimethylbenzenes) |
| C12 | 16.73 (100% naphthalenes) |
| C13-C15 | 2.8 |
| C16 | 3.08 |
| C20+ | 1.28 (corn oil) |

As shown, the vapor phase olefin content was about 80% and the aromatic content in the liquid phase was about 7000.

### Example 8: Single Stage Reactor (Distillers Grade Corn Oil+EtOH+DI Water Feed)

Single Stage reactor configuration: Reaction Conditions: Feed pre-heater=180° C., Reactor T=485° C. in reactor, Distillers Grade Corn Oil flow=0.03 ml/min, EtOH/DI water (65/35%) Flow=0.15 ml/min; WHSV=2.16 (corn oil+EtOH basis), Nitrogen=10 ml/min, P=0 bar; Catalysts—Boron/Phosphor doped ZSM-5 zeolite (SiO₂/AlO₃ ratio=90).

Single pass reactor effluent composition and corresponding weight percent of total as a percent of corn oil mass fed (Table 7A shows the vapor phase and Table 7B shows the liquid phase).

TABLE 7A
(Vapor phase = 58% of Distillers Grade Corn Oil +
EtOH mass fed), CO₂/CO not measured

| Component | Vapor GC Area % |
|---|---|
| ethylene | 51.48 |
| methane | 0.23 |
| ethane | 0.82 |
| propylene | 24.46 |
| propane | 2.36 |
| isobutane | 1.79 |
| butane | 0.59 |
| butadiene | 0.02 |
| trans-2-butene | 2.75 |
| cis-2-butene | 2.00 |
| n-butene + (isobutylene) iC4 | 7.93 |
| 2-pentene (mixed isomers) | 1.15 |
| 1-pentene | 0.28 |
| pentane | 0.14 |
| 3-methyl-1-butene (3M1B) | 0.22 |
| 2-methyl-1-butene (2M1B) | 1.14 |
| 2-methyl-2-butene (2M2B) | 2.13 |
| isopentane | 0.54 |
| Total Saturates | 6.24 |

TABLE 7B
(Liquid phase = 42% of Corn Oil + EtOH mass fed)

| Carbon Number | GC Area % - Liquid Composition |
|---|---|
| C2 | 0 |
| C3 | 0.17 |
| C4 | 1.66 |
| C5-C7 | 14.4 (33% benzene) |
| C8 | 23.4 (77% toluene) |
| C9-C11 | 38.8 (76% xylenes, 24% trimethylbenzenes) |
| C12 | 18.1 (100% naphthalenes) |
| C13-C15 | 3.56 |
| C16 | 0 |
| C20+ | 0 |

As shown, the vapor phase olefin content was about 80 and the aromatic content in the liquid phase was about 800.

### Example 9: Single Stage Reactor—Canola Oil+DI Water Feed

Single Stage reactor configuration: Reaction Conditions: Feed pre-heater=180° C., Reactor T=480° C. in reactor, Canola Oil flow=0.20 ml/min, DI water flow=0.10 ml/min; WHSV=4.43 (canola oil basis), Nitrogen=10 ml/min, P=0 bar; Catalysts—Boron/Phosphor doped ZSM-5 zeolite (SiO₂/AlO₃ ratio=90).

Single pass reactor effluent composition and corresponding weight percent of total as a percent of canola oil+water mass fed (Table 8A shows the vapor phase and Table 8B shows the liquid phase).

TABLE 8A
(Vapor phase = 33% of Canola Oil +
DI water mass fed), CO₂/CO not measured

| Component | Vapor GC Area % |
|---|---|
| ethylene | 9.91 |
| methane | 0.4 |
| ethane | 0.61 |
| propylene | 32.16 |
| propane | 2.99 |
| isobutane | 2.3 |
| butane | 1.35 |
| butadiene | 0.16 |
| trans-2-butene | 7.88 |
| cis-2-butene | 5.91 |
| n-butene + (isobutylene) iC4 | 17.82 |
| 2-pentene (mixed isomers) | 3.63 |
| 1-pentene | 0.75 |
| pentane | 0.2 |
| 3-methyl-1-butene (3M1B) | 0.58 |
| 2-methyl-1-butene (2M1B) | 3.96 |
| 2-methyl-2-butene (2M2B) | 8.12 |
| isopentane | 1.29 |
| Total Saturates | 8.74 |

TABLE 8B
(Liquid phase = 67% of Canola Oil + DI water mass fed)

| Carbon Number | GC Area % - Liquid Composition |
|---|---|
| C2 | 0 |
| C3 | 0.68 |
| C4 | 2.75 |
| C5-C7 | 18.2 (45% benzene) |
| C8 | 24.9 (84% toluene) |
| C9-C11 | 32.8 (78% xylenes, 22% trimethylbenzenes) |
| C12 | 14.55 (100% naphthalenes) |
| C13-C15 | 1.67 |
| C16 | 1.57 |
| C20+ | 2.2 |

As shown, the vapor phase olefin content was about 750 and the aromatic content in the liquid phase was about 750.

### Example 10: Single Stage Reactor—Canola Oil+EtOH+DI Water Feed

Single Stage reactor configuration: Reaction Conditions: Feed pre-heater=180° C., Reactor T=480° C. in reactor, Canola Oil flow=0.10 ml/min, EtOH/I water (50/50%) Flow=0.20 ml/min; WHSV=4.35 (canola oil+EtOH basis), Nitrogen=10 ml/min, P=0 bar; Catalysts—Boron/Phosphor doped ZSM-5 zeolite (SiO₂/AlO₃=90).

Single pass reactor effluent composition and corresponding weight percent of total as a percent of canola oil+EtOH/water mass fed (Table 9A shows the vapor phase and Table 9B shows the liquid phase).

TABLE 9A
(Vapor phase = 43% of Canola Oil + EtOH/water
mass fed), CO₂/CO not measured

| Component | Vapor GC Area % |
|---|---|
| ethylene | 36.43 |
| methane | 0.27 |
| ethane | 0.67 |
| propylene | 24.56 |
| propane | 2.97 |
| isobutane | 2.96 |
| butane | 1.24 |
| butadiene | 0 |
| trans-2-butene | 4.6 |
| cis-2-butene | 3.41 |
| n-butene + (isobutylene) iC4 | 11.46 |
| 2-pentene (mixed isomers) | 2.34 |
| 1-pentene | 0.81 |
| pentane | 0.3 |
| 3-methyl-1-butene (3M1B) | 0.33 |
| 2-methyl-1-butene (2M1B) | 1.96 |
| 2-methyl-2-butene (2M2B) | 4.03 |
| isopentane | 1.21 |
| Total Saturates | 9.35 |

TABLE 9B
(Liquid phase = 57% of Canola Oil + EtOH/water
mass fed), CO₂/CO not measured

| Carbon Number | GC Area % - Liquid Composition |
|---|---|
| C2 | 0 |
| C3 | 0.31 |
| C4 | 1.3 |
| C5-C7 | 14.2 (43% benzene) |
| C8 | 24.5 (79% toluene) |
| C9-C11 | 38.8 (74% xylenes, 26% trimethylbenzenes) |
| C12 | 16.93 (100% naphthalenes) |
| C13-C15 | 2.17 |
| C16 | 1.11 |
| C20+ | 0.76 |

As shown, the vapor phase olefin content was about 7500 and the aromatic content in the liquid phase was about 800%.

### Example 11: Single Stage Reactor—Coconut Oil+EtOH+DI Water Feed

Single Stage reactor configuration: Reaction Conditions: Feed pre-heater=180° C., Reactor T=450° C. in reactor, Coconut Oil flow=0.10 ml/min, EtOH/DI water (65/35%) Flow=0.15 ml/min; WHSV=3.36 (coconut oil+EtOH basis), Nitrogen=10 ml/min, P=0 bar; Catalysts—Boron/Phosphor doped ZSM-5 zeolite (SiO₂/AlO₃ ratio=90)+Zr-doped [symbol] -alumina.

Single pass reactor effluent composition and corresponding weight percent of total as a percent of coconut oil+EtOH/water mass fed (Table 10 shows the vapor phase and the liquid phase was not analyzed).

TABLE 10
(Vapor phase = 45% of Coconut Oil + EtOH/water
mass fed), CO₂/CO not measured

| Component | Vapor GC Area % |
|---|---|
| ethylene | 39.73 |
| methane | 0.14 |
| ethane | 0.61 |
| propylene | 24.22 |
| propane | 3.53 |
| isobutane | 3.91 |
| butane | 1.55 |
| butadiene | 0 |
| trans-2-butene | 4.17 |
| cis-2-butene | 3.02 |
| n-butene + (isobutylene) iC4 | 11.83 |
| 2-pentene (mixed isomers) | 1.29 |
| 1-pentene | 0.32 |
| pentane | 0.39 |
| 3-methyl-1-butene (3M1B) | 0.27 |
| 2-methyl-1-butene (2M1B) | 1.39 |
| 2-methyl-2-butene (2M2B) | 2.39 |
| isopentane | 1.24 |
| Total Saturates | 11.23 |

As shown, the vapor phase olefin content was about 7500.

Although various illustrative aspects are described above, any of a number of changes can be made to various aspects without departing from the teachings herein. For example, the order in which various described process steps are performed can often be changed in alternative aspects, and in other alternative aspects, one or more process steps can be skipped altogether. Optional features of various system and process aspects can be included in some aspects and not in others. Therefore, the foregoing description is provided primarily for exemplary purposes and should not be interpreted to limit the scope of the claims.

The examples and illustrations included herein show, by way of illustration and not of limitation, specific aspects in which the subject matter can be practiced. As mentioned, other aspects can be utilized and derived there from, such that structural and logical substitutions and changes can be made without departing from the scope of this disclosure. Such aspects of the inventive subject matter can be referred to herein individually or collectively by the term “invention” merely for convenience and without intending to voluntarily limit the scope of this application to any single invention or inventive concept, if more than one is, in fact, disclosed. Thus, although specific aspects have been illustrated and described herein, any arrangement calculated to achieve the same purpose can be substituted for the specific aspects shown. This disclosure is intended to cover any and all adaptations or variations of various aspects. Combinations of the above aspects, and other aspects not specifically described herein, will be apparent to those of skill in the art upon reviewing the above description. Use of the term “based on,” herein and in the claims is intended to mean, “based at least in part on,” such that an unrecited feature or element is also permissible.

The subject matter described herein can be embodied in systems, apparatus, methods, and/or articles depending on the desired configuration. The aspects set forth in the foregoing description do not represent all aspects consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the aspects described herein can be directed to various combinations and subcombinations of the disclosed features and/or combinations and subcombinations of several further features disclosed herein. In addition, the logic flows depicted in the accompanying figures and/or described herein do not necessarily require the particular order shown, or sequential order, to achieve desirable results. Other aspects can be within the scope of the following claims.

1. A process for producing one or more olefins, the process comprising:
contacting an input stream with one or more catalysts in at least one reactor to form an output stream comprising the one or more olefins, the input stream comprising one or more bio-oils, and a first of the one or more catalysts comprising a first zeolite and one or more first dopants;
wherein the at least one reactor is at a temperature from about 300° C. to about 600° C., a gauge pressure from about 1 to about 10 bar, and a weight hourly space velocity (WHSV) from about 0.5 h⁻¹ to about 10 h⁻¹.

2. The process of claim 1, wherein the at least one reactor comprises two or more catalyst beds.

3. The process of claim 1, wherein the at least one reactor comprises one or more catalyst beds, wherein the process further comprises impregnating at least one catalyst bed of the one or more catalysts beds with the first catalyst.

4. The process of claim 2, wherein the two or more catalysts beds are stacked relative to each other within the single reactor.

5. The process of claim 1, wherein the at least one reactor is a single bed reactor.

6.-8. (canceled)

9. The process of claim 1, wherein the one or more olefins comprises C₂-C₅ olefins.

10. The process of claim 9, wherein the one or more olefins comprises a predominant first olefin, wherein the predominant first olefin is propylene.

11. (canceled)

12. The process of claim 9, wherein the one or more C₂-C₅ olefins are present in the output stream in an amount that is from about 65 wt. % to about 85 wt. % of the total amount of unsaturated hydrocarbons present in the output stream.

13. (canceled)

14. The process of claim 1, wherein the output stream further comprises one or more aromatics.

15. (canceled)

16. The process of claim 1, wherein the one or more dopants comprises boron, phosphor, or a combination thereof.

17.-20. (canceled)

21. The process of claim 1, wherein the first zeolite comprises a ZSM-5 zeolite.

22. The process of claim 1, wherein the output stream further comprises saturates, wherein a total amount of saturates present in the output stream does not exceed about 20 wt. % of the output stream.

23.-24. (canceled)

25. The process of claim 1, further comprising, prior to contacting the input stream with the one or more catalysts, combining the input stream with water, one or more C₁-C₅ alcohols, or water and one or more C₁-C₅ alcohols.

26. The process of claim 25, wherein the one or more C₁-C₅ alcohols comprises ethanol, methanol, isobutanol, or any combination thereof.

27.-28. (canceled)

29. The process of claim 25, wherein the one or more C₁-C₅ alcohols comprises a mixture of at least C₄ alcohols, a mixture of C₅ alcohols, or a mixture of C₄ and C₅ alcohols.

30. (canceled)

31. The process of claim 1, further comprising removing at least a portion of C₂ olefins, C4 olefins, C5 olefins, or any combination thereof from the output stream.

32.-33. (canceled)

34. The process of claim 1, further comprising removing at least a portion of aromatics from the output stream.

35.-38. (canceled)

39. The process of claim 1, wherein a second of the one or more catalysts comprises a second zeolite and one or more second dopants.

40. (canceled)

41. The process of claim 1, wherein a second of the one or more catalysts comprises a doped or undoped alumina catalyst, and optionally, wherein the doped alumina catalyst comprises, in neutral or ionic form, zirconium, titanium, tungsten, silicon, fluorine, or any combination thereof.

42. (canceled)

43. The process of claim 1, wherein a second of the one or more catalysts comprises a silicated [symbol] -alumina, zirconated [symbol] -alumina, titanated [symbol] -alumina, niobium [symbol] -alumina, or fluorinated [symbol] -alumina, an undoped [symbol] -alumina, an undoped zeolite, a silica alumina catalyst, or any combination thereof.

44.-89. (canceled)
